(19) United States
(12) Patent Application Publication
(10) Pub. No.: US 2020/0023001 A1
(43) Pub. Date: Jan. 23, 2020

(54) GlycoFix (Structurally And Functionally Repaired Endothelial Glycocalyx)

(71) Applicant: Northeastern University, Boston, MA (US)

(72) Inventors: Eno Essien Ebong, Milton, MA (US); Solomon A. Mensah, Malden, MA (US); Ming Cheng, Boston, MA (US); Ronodeep Mitra, Boston, MA (US)

(21) Appl. No.: 16/517,274

(22) Filed: Jul. 19, 2019

Related U.S. Application Data
(60) Provisional application No. 62/701,561, filed on Jul. 20, 2018.

(51) Int. Cl. A61K 31/737; A61K 31/661; A61K 9/00; A61P 9/10

(52) U.S. Cl. CPC A61K 31/737; A61P 9/10; A61K 9/0024; A61K 31/661

(57) ABSTRACT

Provided herein are compositions comprising heparan sulfate, or a pharmaceutically acceptable salt thereof, and sphingosine-1-phosphate, or a pharmaceutically acceptable salt thereof. Such compositions are useful in a variety of methods, including methods of regenerating endothelial glycocalyx, preventing endothelial glycocalyx degradation and treating vascular disease.

### RELATED APPLICATION

This application claims the benefit of U.S. Provisional Application No. 62/701,561, filed Jul. 20, 2018. The entire teachings of the above application are incorporated herein by reference.

### GOVERNMENT FUNDING

This invention was made with government support under Grant No. K01 HL125499 awarded by the National Institutes of Health. The government has certain rights in the invention.

This invention was made with government support under Grant No. DGE 1451070 awarded by the National Science Foundation. The government has certain rights in this invention.

This invention was made with government support under Grant No. DGE 0965843 awarded by the National Science Foundation. The government has certain rights in this invention.

### BACKGROUND

Vasculoprotective endothelial cells (ECs) exhibit a number of behaviors that include regulation of vascular permeability and inflammation along with control of vascular tone. An important contributor to these functions is the EC membrane-anchored, mesh-like extracellular matrix: a sugar coat known as the glycocalyx (GCX). The location and anchoring of the GCX enables EC sensitivity to extracellular microenvironment conditions, which ECs transduce into specific biological behaviors in a temporal and spatial manner.

GCX composition is actively regulated by EC through continuous shedding and synthesis. At healthy vascular sites, shedding and synthesis are balanced, and the intact GCX can transmit signals into the cell to trigger vasculoprotective cell function. At diseased vascular sites, shedding exceeds synthesis, leading to increased GCX permeability and/or reduced thickness. Consequently, cell signal transmission becomes dysfunctional in a manner that is permissive of disease.

Accordingly, there is a need for compositions, methods, kits and other articles that can regenerate shed GCX, reverse dysfunctional cell signaling and/or treat or prevent disease.

### SUMMARY

Provided herein are compositions, methods, kits and other articles that, at least in some embodiments, support structural and functional repair of endothelial glycocalyx.

Provided herein is a composition, comprising heparan sulfate, or a pharmaceutically acceptable salt thereof, and sphingosine-1-phosphate, or a pharmaceutically acceptable salt thereof.

Also provided herein is a method of regenerating glycocalyx of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof.

Also provided herein is a method of increasing expression of a connexin by a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof.

Also provided herein is a method of increasing stability of the plasma membrane of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof.

Also provided herein is a method of improving gap junction function of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof.

Also provided herein is a method of increasing intercellular communication between two or more cells (e.g., endothelial cells), comprising contacting at least one of the two or more cells with an effective amount of heparan sulfate, or a pharmaceutically acceptable salt thereof, and an effective amount of sphingosine-1-phosphate, or a pharmaceutically acceptable salt thereof.

Also provided herein is a method of inhibiting glycocalyx degradation of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof.

Also provided herein is a method of treating a vascular disease in a subject in need thereof, comprising administering to the subject a therapeutically effective amount of heparan sulfate, or a pharmaceutically acceptable salt thereof, and a therapeutically effective amount of sphingosine-1-phosphate, or a pharmaceutically acceptable salt thereof.

Also provided herein is an implant (e.g., stent, catheter, prosthesis) or vascular graft, comprising heparan sulfate, or a pharmaceutically acceptable salt thereof, and sphingosine-1-phosphate, or a pharmaceutically acceptable salt thereof.

Also provided herein is a kit comprising heparan sulfate, or a pharmaceutically acceptable salt thereof, and sphingosine-1-phosphate, or a pharmaceutically acceptable salt thereof.

In some embodiments, the compositions, methods, kits and other articles described herein have the ability to replace lost heparan sulfate from the endothelial glycocalyx, restore glycocalyx-dependent barrier function, recover glycocalyx-dependent cell-to-cell communication and/or reverse endothelial cell dysfunction. The ability of the compositions, methods, kits and other articles to simultaneously replace endothelial glycocalyx components and restore complicated cellular functions, including the cell-to-cell communication function, which is difficult to recover and is an important part of endothelial functioning, is a particular advantage of embodiments of this invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments.

FIGS. 1A-1B. Scanning electron micrographs show brush-like protrusions from the cell surface. FIG. 1A. No GCX-specific contrast agent was used. Long arrows point to long, thin extracellular fibrillar structures extending from the surface of the rat fat pad endothelial cell (RFPEC) plasma membrane. Arrowheads point to intercellular gaps. FIG. 1B. Ruthenium red was used as a contrast agent. Ruthenium red specifically binds GAGs, and revealed the strong presence of GAGs at the apical surface and at borders of the cell body. Medium length arrows point to ruthenium red at cell borders. Arrowhead points to intercellular gap. Scale bar=10 m.

FIGS. 2A-2G. Heparan sulfate (HS) expression in intact GCX, enzyme-degraded GCX, and repaired GCX. FIG. 2A. Untreated (control) cells show intact HS at baseline conditions (green is HS with blue DAPI staining the cell nucleus). FIG. 2B. With 25 μIU/ml of Hep III, HS is degraded. FIG. 2C. Degraded HS samples were left for 24 hours to allow cells to regenerate HS. FIG. 2D. With the addition of HS at a concentration of 59 μg/ml, there is a significant recovery of HS back to baseline conditions. Scale bar=20 μm, and applies to 2A-2F. FIG. 2E. Adding 10 μM sphingosine-1-phosphate (S1P) to enzyme treated samples significantly recovered the expression of HS in GCX, back to baseline conditions. FIG. 2F. Combined treatment of exogenous HS and S1P affected HS expression by restoring baseline conditions. FIG. 2G. Quantification of HS in control, Hep III-treated, self-recovery, and artificial recovery conditions. ANOVA and Tukey post hoc test showed statistical significance, as noted in the plot and summarized in the table. The results of various treatment conditions were compared to either control or enzyme (Hep III) conditions.

FIGS. 3A-3G. Images and quantification of Cx43 after various modes of GCX recovery. FIGS. 3A-3F. Representative images of Cx43 expression at the monolayer level, with insets clarifying Cx43 distribution at cell borders. FIG. 3A. Untreated sample. FIG. 3B. After Hep III treatment, Cx43 distribution was reduced. FIG. 3C. After enzyme treatment, samples were left to self-recover their Cx43 over 24 hours. FIG. 3D. Cx43 distribution was partially restored after addition of 59 μg/ml exogenous HS to counteract enzyme treatment. Scale bar=100 μm in low magnification image and 2.5 μm in high magnification image, and both scale bars apply to FIGS. 3A-3F. FIG. 3E. Addition of 10 μM S1P alone did not show a significant restoration of Cx43 expression in comparison to enzyme-treated samples. FIG. 3F. Combined treatment of HS and S1P after Hep III treatment resulted in expression of Cx43 that matched controls and was greater than in enzyme-treated samples. FIG. 3G. Quantification of Cx43 distribution at cell borders of RFPEC for control, Hep III-treated, self-recovery, and artificial recovery conditions. ANOVA and Tukey post hoc test showed statistical significance, as noted in the plot and summarized in the table. The results of various treatment conditions were compared to either control or enzyme (Hep III) conditions.

FIGS. 4A-4G. Quantification of Lucifer Yellow dye transfer after various modes of GCX recovery. FIG. 4A. Control. FIG. 4B. With 25 IU/ml Hep III treatment, the movement of Lucifer Yellow dye across cell junctions was impaired. FIG. 4C. Self-recovery of HS, after enzymatic degradation, allowed dye transfer across cell junctions at near baseline levels. FIG. 4D. Not much Lucifer Yellow dye transfer improvement was observed after recovery of lost HS by addition of 59 μg/ml HS. Scale bar=100 μm and applies to FIGS. 4A-4F. FIG. 4E. Addition of 10 μM S1P also did not improve cell-to-cell communication. FIG. 4F. A combined treatment of RFPEC with both HS and S1P resulted in an increase in Lucifer Yellow dye transfer, reversing the effects of enzyme treatment. FIG. 4G. Quantification of Lucifer Yellow flux across connexin-containing gap junctions in RFPEC in control, Hep III-treated, self-recovery, and artificial recovery conditions. ANOVA and Tukey post hoc test showed statistical significance, as noted in the plot and summarized in the table. The results of various treatment conditions were compared to either control or enzyme (Hep III) conditions.

FIGS. 5A-5C. Conceptual hypothesis. FIG. 5A. Inter-endothelial gap junction is fully functional due to intact GCX (green arrows show Lucifer Yellow dye flux) in control conditions, when cells self-recover HS, or when HS is artificially recovered by treatment of cells with combination of exogenous HS and S1P. FIG. 5B. Degraded HS from GCX will cause the dislocation of Cx43 and malfunction of gap junction channels, thereby preventing the movement of Lucifer Yellow dye across inter-endothelial gap junctions (red line indicates the improper functioning or blockage of gap junctions). FIG. 5C. Treatment of enzyme-treated cells with exogenous HS or S1P does not fully reset Cx43 and/or recover intercellular gap junctional communication.

FIG. 6A. Negative control for Cx43: Primary antibody specific to Cx43 was omitted in the immunostaining protocol to confirm the specificity of the antibody. No Cx43 was stained (blue stain indicates DAPI, which stains the cell nucleus).

FIG. 6B. Negative Control for HS: Primary antibody targeting HS was omitted to confirm the specificity of the antibody. No HS was stained as observed both in the en face image and the orthogonal view (blue stain indicates DAPI staining for cell nucleus).

FIG. 7A. To quantify Cx43 coverage of RFPEC monolayer, ImageJ automatically selected nine locations, marked with red crosses, to randomize the cells that were quantified.

FIG. 7B. To quantify gap junction mediated cell communication, cells were scratch-loaded (red-labeled cells only) with Lucifer Yellow dye, the dye spread to neighbors, and the neighboring cells that contained Lucifer yellow dye (green-labeled cells) were counted along lines perpendicular to the scratch.

FIGS. 8A-8E. Size, fluorescence, and toxicity measurements of PEG-AuNP. FIG. 8A. TEM of AuNP showing individual NPs and sample measurements. FIG. 8B. DLS plot indicating an increase in diameter of AuNP after PEGylation. FIG. 8C. Fluorescence peak at 667 nm matches the emission of Alexa Fluor 647 conjugated to PEG-AuNP. FIG. 8D. MTS assays show no PEG-AuNP toxicity to RFPEC up to 1,000 μg/mL for 16 hours. FIG. 8E. Histogram of AuNP size distribution measured from TEM images. Abbreviations: TEM, transmission electron microscope; AuNP, gold NPs; DLS, dynamic light scattering; PEG, polyethelyne glycol; MTS, 3-(4,5-dimethylthiazol-2-yl)-5-(3-carboxymethoxyphenyl)-2-(4-sulfophenyl)-2H-tetrazolium; RFPEC, rat fat pad endothelial cell; NP, nanoparticle.

FIGS. 9A-9F. En face and cross sections of RFPEC in which BSA was immunostained in green and cell nuclei were immunostained in blue. FIG. 9A. Control. FIG. 9B. Low serum. FIG. 9C. Hep III. FIG. 9D. Hep III+HS. FIG. 19E. Quantification of the overall thickness of glycocalyx after various treatments—control, low serum, Hep III, Hep III+HS—depicted by staining BSA adsorbed onto the glycosaminoglycan matrix. The glycocalyx treatments and recovery decreased overall thickness. FIG. 9F. The glycocalyx coverage as seen by BSA immunofluorescence. The various treatments and glycocalyx conditions did not affect overall glycocalyx coverage. *P<0.05 versus control. Abbreviations: RFPEC, rat fat pad endothelial cell; BSA, bovine serum albumin; HEP III, heparinase III; HS, heparan sulfate.

FIGS. 10A-10F. En face and cross sections of RFPEC in which HS was immunostained in green and cell nuclei were immunostained in blue. FIG. 10A. Control. FIG. 10B. low serum. FIG. 10C. Hep III. FIG. 10D. Hep III+HS. FIG. 10E. Thickness measurements of the glycocalyx when observing only the HS component by binding the 10E4 epitope HS antibody to the glycosaminoglycan under control, low serum, Hep III, and Hep III+HS treatments. HS thickness decreased significantly for all treatments. FIG. 10F. HS coverage of RFPEC after various treatments. HS coverage decreased significantly after heparinase III treatment and recovered after HS addition. *P<0.05 versus control and **P<0.05 versus Hep III. Abbreviations: Hep III, heparinase III; HS, heparan sulfate; RFPEC, rat fat pad endothelial cell.

FIGS. 11A-11C. Uptake and localization differences between glycocalyx conditions. FIG. 11A. Confocal cross sections of PEG-AuNP (red) in RFPEC under various glycocalyx conditions. FIG. 11B. Relative fluorescent AuNP uptake by quantifying the amount of red fluorescence in cross-sectional images and normalizing to controls. *P<0.05. FIG. 11C. Localization of PEG-AuNP within the cross sections vary between dysfunctional types. Red fluorescence was measured with respect to cell height and consolidated in a histogram. Abbreviations: AuNP, gold NPs; PEG, polyethelyne glycol; RFPEC, rat fat pad endothelial cell; Hep III, heparinase III; HS, heparan sulfate; GCX, glycocalyx.

### DETAILED DESCRIPTION

A description of example embodiments follows.

### Compositions, Kits and Other Articles

Provided herein is a composition (e.g., a pharmaceutical composition), comprising heparan sulfate, or a pharmaceutically acceptable salt thereof, and sphingosine-1-phosphate, or a pharmaceutically acceptable salt thereof.

Heparan sulfate (HS) is the most common glycocalyx glycosaminoglycan, and is anchored to endothelial cells via transmembrane proteoglycans. Together, a layer of glycosaminoglycans, including HS, and a layer of proteoglycans form the glycocalyx, a coating on the endothelium responsible for ensuring proper functioning of the endothelium. Exogenous heparan sulfate can be extracted from porcine mucosal tissue and, typically, exogenous heparan sulfate thus obtained has an average molecular weight of about 15 kDa. Thus, in some embodiments, heparan sulfate is exogenous heparan sulfate. In some embodiments, heparan sulfate has an average molecular weight of about 15 kDa. In some embodiments, heparan sulfate is porcine mucosal heparan sulfate (e.g., porcine mucosal heparan sulfate with an average molecular weight of about 15 kDa).

“Exogenous,” as used herein with respect to HS, refers to HS extracted from its native environment. Exogenous HS may be used, for example, to replace and/or replenish degraded HS in a different organism (as when porcine mucosal HS is introduced into rat fat pad endothelial cells, for example) or at a different site within an organism (as in a graft, for example).

Sphingosine-1-phosphate (S1P) is a bioactive sphingolipid metabolite that enhances cellular responses including cell growth, survival, migration and decreases in endothelial layer permeability. It is thought to work by binding to a specific subfamily of G-protein-coupled receptors.

GlycoFix is a chemical formulation comprised of exogenous porcine mucosal heparan sulfate and sphingosine-1-phosphate that can be used for the repair of degraded endothelial glycocalyx and to restore intercellular communication. Endothelial glycocalyx shedding has been attributed to the onset of diseases of the blood vessels, such as atherosclerosis, and the shedding represents a loss of endothelial cell protection against harmful molecules and cells flowing in the blood. The loss of glycocalyx causes endothelial cell dysfunction; GlycoFix aims to reverse this through recovery of the glycocalyx layer, thereby delaying the onset of atherosclerosis and related disease conditions.

In an example embodiment, GlycoFix is a blend of the following two compounds:
1. Heparan Sulphate extracted exogenously from porcine mucosal tissue with average molecular weight of approximately 15 kDa; and
2. Sphingosine-1-Phosphate, a bioactive sphingolipid metabolite that enhances cellular responses including cell growth, survival, migration and the decrease in endothelial layer permeability.

As used herein, the term “pharmaceutically acceptable salt” refers to those salts which are, within the scope of sound medical judgment, suitable for use in contact with the tissues of a subject without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, the relevant teachings of which are incorporated herein by reference in their entirety. Pharmaceutically acceptable salts include salts derived from suitable inorganic and organic acids and inorganic and organic bases. Typically, a pharmaceutically acceptable salt of heparan sulfate will be a salt derived from a suitable inorganic or organic base, or a base addition salt.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid, or by using other methods used in the art, such as ion-exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N+((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Mono- or poly- (e.g., di- or tri-) salts can be formed, and such salts can exist in either a hydrated, solvated or substantially anhydrous form.

Heparan sulfate, sphingosine-1-phospate and their pharmaceutically acceptable salts can also exist as various “solvates” or “hydrates,” and such solvates and hydrates are within the scope of this invention unless the context clearly dictates otherwise. A “hydrate” is a compound that exists in a composition with one or more water molecules. The composition can include water in stoichiometic quantities, such as a monohydrate or a dihydrate, or can include water in random amounts. A “solvate” is similar to a hydrate, except that a solvent other that water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Also provided herein is a kit comprising (e.g., an effective amount of) heparan sulfate, or a pharmaceutically acceptable salt thereof (e.g., a composition comprising heparan sulfate, or a pharmaceutically acceptable salt thereof), and (e.g., an effective amount of) sphingosine-1-phosphate, or a pharmaceutically acceptable salt thereof (e.g., a composition comprising sphingosine-1-phosphate, or a pharmaceutically acceptable salt thereof). In some embodiments, the kit further comprises instructions (e.g., written instructions) for administering the heparan sulfate, or a pharmaceutically acceptable salt thereof, and sphingosine-1-phosphate, or a pharmaceutically acceptable salt thereof, to a subject for treatment of a vascular disease (e.g., atherosclerosis). When heparan sulfate, or a pharmaceutically acceptable salt thereof, and sphingosine-1-phosphate, or a pharmaceutically acceptable salt thereof, are provided in a kit, the two can be administered concurrently, or heparan sulfate, or a pharmaceutically acceptable salt thereof, can be administered before or after sphingosine-1-phosphate, or a pharmaceutically acceptable salt thereof.

In some embodiments, a pharmaceutical composition further comprises one or more pharmaceutically acceptable carriers.

“Pharmaceutically acceptable carrier” refers to a non-toxic carrier, diluent or excipient that does not destroy the pharmacological activity of the therapeutic agent(s) (e.g., heparan sulfate, or a pharmaceutically acceptable salt thereof; sphingosine-1-phosphate, or a pharmaceutically acceptable salt thereof) with which it is formulated and is nontoxic when administered in doses sufficient to deliver a therapeutic amount of the therapeutic agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions described herein may be administered orally, parenterally (including subcutaneously, intramuscularly, intravenously and intradermally), by inhalation spray, topically, rectally, nasally, buccally, vaginally or via an implanted reservoir. In some embodiments, a composition is orally administrable. In some embodiments, a composition is intravenously administrable.

The term “parenteral,” as used herein, includes subcutaneous, intracutaneous, intravenous, intramuscular, intraocular, intravitreal, intra-articular, intra-arterial, intra-synovial, intrasternal, intrathecal, intralesional, intrahepatic, intraperitoneal, intralesional and intracranial injection or infusion techniques.

Compositions provided herein can be orally administered in any orally acceptable dosage form including, but not limited to, capsules, tablets, aqueous suspensions, dispersions and solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions and/or emulsions are required for oral use, the active ingredient can be suspended or dissolved in an oily phase and combined with emulsifying and/or suspending agents. If desired, certain sweetening, flavoring or coloring agents may also be added.

In some embodiments, an oral formulation is formulated for immediate release. In some embodiments, an oral formulation is formulated for sustained/delayed release.

Solid dosage forms for oral administration include capsules, tablets, pills, powders, and granules. In such solid dosage forms, the therapeutic agent(s) is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders, such as carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium salts, (g) wetting agents, such as acetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Liquid dosage forms for oral administration include pharmaceutically acceptable emulsions, microemulsions, solutions, suspensions, syrups, and elixirs. In addition to the therapeutic agent(s), the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, or mixtures thereof. Besides inert diluents, the oral compositions can also include adjuvants, such as wetting agents, emulsifying and suspending agents, sweetening, flavoring, coloring, perfuming, and preservative agents.

Compositions suitable for buccal or sublingual administration include tablets, lozenges and pastilles, wherein the therapeutic agent(s) is formulated with a carrier such as sugar and acacia, tragacanth, or gelatin and glycerin.

Solid compositions of a similar type may also be employed as fillers in soft and hard-filled gelatin capsules using excipients such as lactose or milk sugar, as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the therapeutic agent(s) only, or preferentially, in a certain part of the intestinal tract, optionally, in a delayed manner. Examples of embedding compositions that can be used include polymeric substances and waxes.

A composition can also be in micro-encapsulated form. In such solid dosage forms, the therapeutic agent(s) can be admixed with at least one inert diluent such as sucrose, lactose or starch. Such dosage forms can also comprise, as is normal practice, additional substances other than inert diluents, e.g., tableting lubricants and other tableting aids such a magnesium stearate and microcrystalline cellulose.

Compositions for oral administration may be designed to protect the therapeutic agent(s) against degradation as it passes through the alimentary tract, for example, by an outer coating of the formulation on a tablet or capsule.

In another embodiment, a composition can be provided in an extended (or “delayed” or “sustained”) release composition. This delayed-release composition comprises the therapeutic agent(s) in combination with a delayed-release component. Such a composition allows targeted release of a provided agent into the lower gastrointestinal tract, for example, into the small intestine, the large intestine, the colon and/or the rectum. In certain embodiments, a delayed-release composition further comprises an enteric or pH-dependent coating, such as cellulose acetate phthalates and other phthalates (e.g., polyvinyl acetate phthalate, methacrylates (Eudragits)). Alternatively, the delayed-release composition provides controlled release to the small intestine and/or colon by the provision of pH-sensitive methacrylate coatings, pH-sensitive polymeric microspheres, or polymers which undergo degradation by hydrolysis. The delayed-release composition can be formulated with hydrophobic or gelling excipients or coatings. Colonic delivery can further be provided by coatings which are digested by bacterial enzymes such as amylose or pectin, by pH-dependent polymers, by hydrogel plugs swelling with time (Pulsincap), by time-dependent hydrogel coatings and/or by acrylic acid linked to azoaromatic bonds coatings.

Compositions described herein can also be administered subcutaneously, intraperitoneally or intravenously. Compositions described herein for intravenous, subcutaneous, or intraperitoneal injection may contain an isotonic vehicle such as sodium chloride injection, Ringer's injection, dextrose injection, dextrose and sodium chloride injection, lactated Ringer's injection, or other vehicles known in the art.

Compositions described herein can also be administered in the form of suppositories for rectal administration. These can be prepared by mixing the therapeutic agent(s) with a suitable non-irritating excipient that is solid at room temperature but liquid at rectal temperature and, therefore, will melt in the rectum to release the drug. Such materials include cocoa butter, beeswax and polyethylene glycols.

Compositions described herein can also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Transdermal patches can also be used for topical applications.

For other topical applications, the compositions can be formulated in a suitable ointment containing the therapeutic agent(s) suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water and penetration enhancers. Alternatively, compositions can be formulated in a suitable lotion or cream containing the therapeutic agent(s) suspended or dissolved in one or more pharmaceutically acceptable carriers. Alternatively, the composition can be formulated with a suitable lotion or cream containing the therapeutic agent(s) suspended or dissolved in a carrier with suitable emulsifying agents. In some embodiments, suitable carriers include, but are not limited to, mineral oil, sorbitan monostearate, polysorbate 60, cetyl esters wax, cetearyl alcohol, 2-octyldodecanol, benzyl alcohol and water. In other embodiments, suitable carriers include, but are not limited to, mineral oil, sorbitan monostearate, polysorbate 60, cetyl esters wax, cetearyl alcohol, 2-octyldodecanol, benzyl alcohol, water and penetration enhancers.

For ophthalmic use, compositions can be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic use, the compositions can be formulated in an ointment such as petrolatum.

Compositions can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

The compositions can be in the form of a sterile injectable preparation, for example, as a sterile injectable aqueous or oleaginous suspension. This suspension can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms can also be used for the purposes of formulation.

The amount of a therapeutic agent(s) that can be combined with the carrier materials to produce a composition in a single dosage form will vary depending upon the host treated, the particular mode of administration and the activity of the agent employed. Preferably, compositions should be formulated so that a dosage of from about 0.01 mg/kg to about 100 mg/kg body weight/day of the agent can be administered to a subject receiving the composition.

The desired dose may conveniently be administered in a single dose or as multiple doses administered at appropriate intervals such that, for example, the agent is administered 1, 2, 3, 4, 5, 6 or more times per day. The daily dose can be divided, especially when relatively large amounts are administered, or as deemed appropriate, into several, for example 2, 3, 4, 5, 6 or more, administrations.

It should also be understood that a specific dosage and treatment regimen for any particular subject will depend upon a variety of factors, including the activity of the specific agent employed, the age, body weight, general health, sex, diet, time of administration, rate of excretion, drug combination, the judgment of the treating physician and the severity of the particular disease being treated. The amount of a therapeutic agent(s) in the composition will also depend upon the particular therapeutic agent(s) in the composition.

The compositions described herein can, for example, be administered by injection, intravenously, intraarterially, intraocularly, intravitreally, subdermally, orally, buccally, nasally, transmucosally, topically, in an ophthalmic preparation, or by inhalation, in a dosage ranging from about 0.5 mg/kg to about 100 mg/kg of body weight or, alternatively, in a dosage ranging from about 1 mg/dose to about 1000 mg/dose, every 4 to 120 hours, or according to the requirements of the particular therapeutic agent. Typically, the compositions will be administered from about 1 to about 6 (e.g., 1, 2, 3, 4, 5 or 6) times per day or, alternatively, as an infusion (e.g., a continuous infusion) or bolus. The amount of therapeutic agent that can be combined with a carrier material to produce a single dosage form will vary depending upon the host treated and the particular mode of administration. A typical preparation will contain from about 5% to about 95% therapeutic agent(s) (w/w). Alternatively, a preparation can contain from about 20% to about 80% therapeutic agent(s) (w/w).

Doses lower or higher than those recited above may be required. Specific dosage and treatment regimens for any particular subject will depend upon a variety of factors, including the activity of the specific agent employed, the age, body weight, general health status, sex and/or diet of the subject, time of administration, rate of excretion, drug combination, the severity and course of the disease, condition or symptoms, the subject's disposition to the disease, condition or symptoms, and the judgment of the treating physician.

The appropriate dosage for increased efficacy of GlycoFix is dependent on body weight and the seriousness of the pathology. In one embodiment, dosage is a combined treatment of approximately 10 μM of sphingosine-1-phosphate and approximately 59 μg/ml of heparan sulfate.

It is also believed that the compositions and/or kits described herein (e.g., GlycoFix) can be applied to nanomedicine or as a coating on stents and catheters to prevent localized endothelial glycocalyx degradation during disease progression or during vascular recovery after equipment deploy.

Accordingly, also provided herein is an implant (e.g., a stent, a catheter, a prosthesis) comprising exogenous heparan sulfate, or a pharmaceutically acceptable salt thereof, and sphingosine-1-phosphate, or a pharmaceutically acceptable salt thereof. In some aspects of an implant, the heparan sulfate, or a pharmaceutically acceptable salt thereof, and sphingosine-1-phosphate, or a pharmaceutically acceptable salt thereof, are coated on a surface of the implant.

Also provided herein is a vascular graft comprising exogenous heparan sulfate, or a pharmaceutically acceptable salt thereof, and sphingosine-1-phosphate, or a pharmaceutically acceptable salt thereof.

In some embodiments, a composition, kit or other article can further include one or more other therapeutic agents. The additional therapeutic agent(s) can be part of a single dosage form, mixed together in a single composition, or the additional therapeutic agent(s) can be intended for separate administration from heparan sulfate, or a pharmaceutically acceptable salt thereof, and/or sphingosine-1-phosphate, or a pharmaceutically acceptable salt thereof, for example, included in a kit. When the additional therapeutic agent(s) is intended for separate administration, it can be administered concurrently with, before or after heparan sulfate, or a pharmaceutically acceptable salt thereof, and/or sphingosine-1-phosphate, or a pharmaceutically acceptable salt thereof.

Examples of other therapeutic agents for inclusion in a kit described herein include statins (e.g., atorvastatin, fluvastatin, lovastatin, pitavastatin, pravastatin, rosuvastatin calcium, simvastatin), fibrates (e.g., gemfibrozil, fenofibrate), niacin, ezetimibe, bile acid sequestrants (e.g., cholestyramine, colestipol, colesevelam), epanova, lovaza, omtryg, vascepa, proprotein convertase subtilism kexin type 9 inhibitors (alirocumab, evolucumab), antiplatelets (e.g., aspirin, clopidogrel, ticagrelor, prasugrel, warfarin), beta blockers (e.g., acebutolol, atenolol, bisoprolol, metoprolol, nadolol, nebivolol, propranolol), angiotensin converting enzyme (ACE) inhibitors (e.g., benazepril, captopril, enalapril, fosinopril, lisinopril, moexipril, perindopril, quinapril, remipril, trandolapril), calcium channel blockers (e.g., amlodipine, diltiazem, felodipine, isradipine, nicardipine, nifedipine, nisoldipine, verapamil) and diuretics (e.g., chlorthalidone, chlorothiazide, hydrochlorothiazide, indapamide, metolazone, amiloride, bumetanide, furosemide, spironolactone, triameterene).

### Methods

GlycoFix is believed to be useful for preventing glycocalyx loss, for example, during (via) intravenous administration of blood or fluids to a subject (e.g., a patient), as a stent coating to aid in vascular endothelial cell recovery after device deploy or to prevent endothelial glycocalyx damage during deploy, and/or for oral administration to a subject (e.g., patient) suffering from excessive shedding of glycocalyx due to increased inflammation caused by the presence of a disease or surgery(ies) (e.g., during surgery).

Accordingly, provided herein is a method of regenerating glycocalyx of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof. Glycocalyx regeneration includes increasing (e.g., restoring to baseline or physiologically normal or beneficial levels) the thickness of the glycocalyx, increasing (e.g., restoring to baseline or physiologically normal or beneficial levels) the coverage of the glycocalyx on a cell, increasing (e.g., restoring to baseline or physiologically normal or beneficial levels) expression of a connexin by a cell, increasing (e.g., restoring to baseline or physiologically normal or beneficial levels) stability of the plasma membrane of a cell, improving (e.g., restoring to baseline or physiologically normal or beneficial levels) gap junction function of a cell, and increasing (e.g., restoring to baseline or physiologically normal or beneficial levels) intercellular communication between two or more cells. Methods of assessing glycocalyx regeneration are known to those of skill in the art and described herein, and include confocal microscopy (e.g., to assess glycocalyx thickness, coverage; to visualize the glycocalyx and its components, such as HS and connexin 43 (Cx43)), immunofluorescence microscopy (e.g., to visualize the glycocalyx and its components, such as HS and connexin 43), scanning electron microscopy (e.g., to visualize the glycocalyx and its components, such as HS) and fluorescence microscopy (e.g., to visualize the glycocalyx and its components, such as HS; to assess gap junction function; to assess dye transfer).

Also provided herein is a method of increasing expression of a connexin (e.g., connexin 43) by a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof. Methods of assessing connexin expression are known to those of skill in the art and described herein, and include confocal microscopy and immunofluorescence microscopy.

Also provided herein is a method of increasing stability of the plasma membrane of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof. Indicators of plasma membrane instability include improper connexin alignment, which can inhibit connexins from combining to form connexons, block connexon docking to form gap junctions, or prevent gap junction gates from opening. Methods of assessing plasma membrane stability are known to those of skill in the art and described herein, and include fluorescence microscopy (e.g., to assess gap junction function; to assess dye transfer).

Also provided herein is a method of improving gap junction function of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof. Indicators of deficient gap junction function include improper connexin alignment, which can inhibit connexins from combining to form connexons, block connexon docking to form gap junctions, or prevent gap junction gates from opening. Improving gap junction function includes increasing plasma membrane stability and increasing proper connexin alignment. Methods of assessing gap junction function are known to those of skill in the art and described herein, and include fluorescence microscopy (e.g., used in association with a dye transfer assay).

Also provided herein is a method of increasing intercellular communication between two or more cells (e.g., two or more endothelial cells), comprising contacting at least one of the two or more cells with an effective amount of heparan sulfate, or a pharmaceutically acceptable salt thereof, and an effective amount of sphingosine-1-phosphate, or a pharmaceutically acceptable salt thereof. Indicators of deficient intercellular communication include improper connexin alignment, which can inhibit connexins from combining to form connexons, block connexon docking to form gap junctions and/or prevent gap junction gates from opening. Improving intercellular communication includes increasing plasma membrane stability and increasing proper connexin alignment. Methods of assessing intercellular communication are known to those of skill in the art and described herein, and include fluorescence microscopy (e.g., used in association with a dye transfer assay).

Also provided herein is a method of inhibiting glycocalyx degradation of a cell (e.g., an endothelial cell), comprising contacting the cell with an effective amount of heparan sulfate, or a pharmaceutically acceptable salt thereof, and an effective amount of sphingosine-1-phosphate, or a pharmaceutically acceptable salt thereof. Glycocalyx degradation includes decreasing (e.g., to below baseline or physiologically normal or beneficial levels) the thickness of the glycocalyx, decreasing (e.g., to below baseline or physiologically normal or beneficial levels) the coverage of the glycocalyx on a cell, decreasing (e.g., to below baseline or physiologically normal or beneficial levels) expression of a connexin by a cell, decreasing (e.g., to below baseline or physiologically normal or beneficial levels) stability of the plasma membrane of a cell, decreasing (e.g., to below baseline or physiologically normal or beneficial levels) gap junction function of a cell, and decreasing (e.g., to below baseline or physiologically normal or beneficial levels) intercellular communication between two or more cells. Inhibition of glycocalyx degradation includes slowing (e.g., the rate of glycocalyx degradation), halting and reversing (e.g., regenerating, for example, to baseline or physiologically normal or beneficial levels) glycocalyx degradation. Methods of assessing glycocalyx degradation are known to those of skill in the art and described herein, and include confocal microscopy (e.g., to assess glycocalyx thickness, coverage; to visualize the glycocalyx and its components, such as HS and connexin 43), immunofluorescence microscopy (e.g., to visualize the glycocalyx and its components, such as HS and connexin 43), scanning electron microscopy (e.g., to visualize the glycocalyx and its components, such as HS) and fluorescence microscopy (e.g., to visualize the glycocalyx and its components, such as HS; to assess gap junction function; to assess dye transfer).

As used herein, the singular forms “a,” “an” and “the” include plural referents unless the context clearly dictates otherwise. Thus, for example, reference to “a” cell can include a plurality of cells. Further, the plurality can comprise more than one of the same cell or a plurality of different cells. Reference to an endothelial cell thus includes a plurality of endothelial cells, as make up the endothelium, for example. Accordingly, any of the methods described herein comprising endothelial cells can also be performed on endothelium.

Thus, in some embodiments, a method of regenerating glycocalyx of a cell is a method of regenerating endothelial glycocalyx. In some embodiments, a method of increasing expression of a connexin by a cell is a method of increasing expression of a connexin by endothelium. In some embodiments, a method of improving gap junction function of a cell is a method of improving endothelial gap junction function. In some embodiments, a method of increasing intercellular communication between two or more cells is a method of increasing endothelial intercellular communication.

In some embodiments, the cell is in a subject (e.g., a subject, such as a human, in need of treatment).

As used herein, the term “subject” refers to an animal. Typically, the animal is a mammal. Examples of subjects include, for example, primates (e.g., humans), cows, sheep, goats, horses, dogs, cats, rabbits, rats, mice, fish, birds and the like. In some embodiments, the subject is a human.

As used herein, a subject (e.g., a human) is “in need of” a treatment if the subject has, or is at risk for developing, a disease or condition described herein (e.g., a vascular disease). Typically, a subject in need of treatment would benefit biologically, medically or in quality of life from such treatment. A skilled medical professional (e.g., physician) can readily determine whether a subject has, or is at risk for developing, a disease or condition described herein.

An “effective amount” is an amount effective, at dosages and for periods of time necessary, to achieve a desired result (e.g., a desired therapeutic result, glycocalyx regeneration, increased expression of a connexin, increased plasma membrane stability, improved gap junction function, increased intercellular communication, inhibition of glycocalyx degradation, etc.). Determination of an effective amount is within the skill of a person of ordinary skill in the art using the guidance provided herein and other methods known in the art. In one embodiment, a combined treatment of approximately 10 μM of sphingosine-1-phosphate and approximately 59 μg/ml of heparan sulfate is an effective amount.

Also provided herein is a method of treating a vascular disease (e.g., atherosclerosis) in a subject in need thereof, comprising administering to the subject a therapeutically effective amount of heparan sulfate, or a pharmaceutically acceptable salt thereof, and a therapeutically effective amount of sphingosine-1-phosphate, or a pharmaceutically acceptable salt thereof.

“Treating,” as used herein, refers to taking steps to deliver a therapy to a subject, such as a mammal, in need thereof (e.g., as by administering to a mammal one or more therapeutic agents). “Treating” includes preventing a disease or condition from occurring in a subject, in particular, when the subject is predisposed to the disease or condition, even if the subject has not yet been diagnosed with having the disease or condition; inhibiting the disease or condition (e.g., as by slowing or stopping its progression or causing regression of the disease or condition); and relieving the symptoms resulting from the disease or condition.

The terms “administer,” “administering,” or “administration,” in connection with any of the therapeutic agents described herein (e.g., heparan sulfate, or a pharmaceutically acceptable salt thereof; sphingosine-1-phosphate, or a pharmaceutically acceptable salt thereof) refer to implanting, absorbing, ingesting, injecting, inhaling, or otherwise introducing a therapeutic agent(s), or a composition thereof, in, on or to a cell, tissue or subject (e.g., human).

A “therapeutically effective amount” is an amount effective, at dosages and for periods of time necessary, to achieve a desired therapeutic result (e.g., treatment, healing, inhibition or amelioration of physiological response or condition, etc.). The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a subject, mode of administration and the ability of a therapeutic, or combination of therapeutics, to elicit a desired response in a subject.

A therapeutically effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Examples of vascular diseases include, but are not limited to, atherosclerosis, a blood clot, a stroke, peripheral artery disease, an aneurysm, a pulmonary embolism, carotid artery disease, arteriovenous malformation, critical limb ischemia, deep vein thrombosis, chronic venous insufficiency, a varicose vein, coronary artery disease, Raynaud's disease and vasculitis. In some embodiments, the vascular disease is atherosclerosis.

An agent described herein (e.g., heparan sulfate, or a pharmaceutically acceptable salt thereof; sphingosine-1-phosphate, or a pharmaceutically acceptable salt thereof) can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the agent and the particular disease to be treated. Administration can be local or systemic as indicated.

One or more therapeutic agents described herein (e.g., heparan sulfate, or a pharmaceutically acceptable salt thereof; sphingosine-1-phosphate, or a pharmaceutically acceptable salt thereof) can also be administered in combination with one or more other therapeutic agents. When administered in a combination therapy, the therapeutic agent(s) can be administered before, after or concurrently with the other therapeutic agent(s). When co-administered simultaneously (e.g., concurrently), the therapeutic agent(s) and other therapeutic agent(s) can be in separate formulations or the same formulation. Alternatively, the therapeutic agent(s) and other therapeutic agent(s) can be administered sequentially, as separate compositions, within an appropriate time frame as determined by a skilled clinician (e.g., a time sufficient to allow an overlap of the pharmaceutical effects of the agents).

Examples of other therapeutic agents useful for treating a vascular disease described herein (e.g., atherosclerosis) include statins (e.g., atorvastatin, fluvastatin, lovastatin, pitavastatin, pravastatin, rosuvastatin calcium, simvastatin), fibrates (e.g., gemfibrozil, fenofibrate), niacin, ezetimibe, bile acid sequestrants (e.g., cholestyramine, colestipol, colesevelam), epanova, lovaza, omtryg, vascepa, proprotein convertase subtilism kexin type 9 inhibitors (alirocumab, evolucumab), antiplatelets (e.g., aspirin, clopidogrel, ticagrelor, prasugrel, warfarin), beta blockers (e.g., acebutolol, atenolol, bisoprolol, metoprolol, nadolol, nebivolol, propranolol), angiotensin converting enzyme (ACE) inhibitors (e.g., benazepril, captopril, enalapril, fosinopril, lisinopril, moexipril, perindopril, quinapril, remipril, trandolapril), calcium channel blockers (e.g., amlodipine, diltiazem, felodipine, isradipine, nicardipine, nifedipine, nisoldipine, verapamil) and diuretics (e.g., chlorthalidone, chlorothiazide, hydrochlorothiazide, indapamide, metolazone, amiloride, bumetanide, furosemide, spironolactone, triameterene).

### EXEMPLIFICATION

This is the first demonstration that degraded endothelial glycocalyx can be regenerated in a manner that effectively restores vasculoprotective endothelial cell function. Specifically, glycocalyx- and communication-deficient endothelial cells were treated with the glycocalyx protector sphingosine-1-phosphate (S1P) combined with exogenous heparan sulfate (HS), a major glycocalyx component. S1P/HS treatment healed the endothelial glycocalyx and, consequently, restored cell communication. Glycocalyx function and cell communication have both been implicated as important players in endothelial cell and vascular physiology. This work has relevance to treatment of cardiovascular diseases and other pathologies as well as functional endothelialization of vascular grafts and other prostheses.

### Example 1. Regeneration of Glycocalyx by Heparan Sulfate and Sphingosine-1-Phosphate Restores Inter-Endothelial Communication

Vasculoprotective endothelium glycocalyx (GCX) shedding plays a critical role in vascular disease. Previous work demonstrated that GCX degradation disrupts endothelial cell (EC) gap junction connexin (Cx) proteins, likely blocking interendothelial molecular transport that maintains EC and vascular tissue homeostasis to resist disease. This work tested the hypothesis that vasculoprotective EC function can be stimulated by replacement of GCX when it is shed. EC with [i] intact heparan sulfate (HS), the most abundant GCX component; [ii] degraded HS; or [iii] HS that was restored after enzyme degradation by cellular self-recovery or artificially, was used. Artificial HS restoration was achieved via treatment with exogenous HS, with or without the GCX regenerator and protector sphingosine-1-phosphate (S1P). In these cells, expression of Cx isotype 43 (Cx43) at EC borders was immunocytochemically examined, and Cx-containing gap junction activity was characterized by measuring interendothelial spread of gap junction-permeable Lucifer Yellow dye. With intact HS, 60% of EC borders expressed Cx43, and dye spread to 2.88±0.09 neighboring cells. HS degradation decreased Cx43 expression to 30%, and reduced dye spread to 1.87±0.06 cells. Cellular self-recovery of HS restored baseline levels of Cx43 and dye transfer. Artificial HS recovery with exogenous HS partially restored Cx43 expression to 46%, and yielded dye spread to only 1.03±0.07 cells. Treatment with both HS and S1P, recovered HS and restored Cx43 to 56% with significant dye transfer to 3.96±0.23 cells. This is the first evidence of GCX regeneration in a manner that effectively restores vasculoprotective EC communication.

### Introduction

The vasculoprotective endothelial cells (ECs) exhibit a number of behaviors that include regulation of vascular permeability and inflammation along with control of vascular tone [1]. An important contributor to these functions is the EC membrane-anchored, mesh-like extracellular matrix; a sugar coat known as the glycocalyx (GCX) [2, 3]. The location and anchoring of the GCX enables EC sensitivity to the extracellular microenvironment conditions [4], which ECs transduce into specific biological behaviors in a temporal and spatial manner [5, 6].

GCX composition is actively regulated by EC through continuous shedding and synthesis [7-12]. At healthy vascular sites, shedding and synthesis are balanced, and the intact GCX can transmit signals into the cell to trigger vasculoprotective cell function [4, 13]. At diseased vascular sites, shedding exceeds synthesis, leading to increased GCX permeability and/or reduced thickness [14-16]. Consequently, cell signal transmission becomes dysfunctional [4] in a manner that is permissive of disease. Regeneration of shed GCX to reverse this dysfunctional cell signaling and prevent disease was the focus of the study described herein. Specifically, the aim was to functionally regenerate the most abundant GCX constituent, heparan sulfate (HS).

Proposed HS regeneration approaches include its replacement, structural stabilization, competitive binding, and synthesis enhancement [17]. Shed HS has been replaced with commercial HS [18] or sulodexide [19, 20], a compound containing both heparan and dermatan sulfate. In other studies, a non-animal heparan sulfate-like polysaccharide, called rhamnan sulfate, was used as a vascular EC HS mimetic [17, 21-23]. For non-vascular applications, semi-synthetic and heparin-like pentosane polysulfate was used as an HS substitute [24]. Compared to HS replacement strategies, structural stabilization of HS is less common, and was only recently achieved through employment of sphingosine-1-phosphate (S1P) [25, 26].

Dermatan sulfate, a constituent of sulodexide, is not among the GAGs that are naturally present in vascular EC GCX [27]. GCX susceptibility to collapse, shedding, or other damage in the presence of destabilizing chemical and mechanical factors [28] was taken into consideration. To minimize GCX instability, HS was combined with S1P to prevent GCX degradation during the regeneration process [25, 26, 29]. In summary, EC with [i] intact HS; [ii]enzymatically degraded HS; and [iii] HS that was artificially regenerated after enzyme degradation was examined.

In previous studies, described above, efficacy of HS regeneration was tested by examining transendothelial permeability and vascular inflammation as markers of HS-dependent cell function [18-20, 25, 30]. Few other important EC functions were tested [20]. To help advance efforts to therapeutically rebuild the GCX in a functional manner, the efficacy of HS regeneration was assessed by probing gap junctions, which are of great interest due to their complexity and mediation of many vasculoprotective EC functions [31-33]. Gap junctions in EC are formed by connexin (Cx) proteins of various isoforms. Cx43 is the most abundantly expressed connexin in cultured EC [34] making it the focus of many previous studies, as well as the present investigation, via immunocytochemical characterization of Cx43 expression at EC borders. Based on prior work by Thi et al. [4], it was expected that HS regeneration would stabilize Cx expression, supporting open gap junction communication. Thi and colleagues noted that HS-bound syndecan and F-actin are connected, and F-actin is linked to the intercellular junction complex via zona occludin 1 (ZO-1) [35], which plays a role in docking the Cx gap junction proteins at the cell membrane [36-38]. Thi et al. also noted coordinated F-actin stress fiber formation and ZO-1 and Cx43 reorganization in response to flow stimuli only when HS is intact and not when it is degraded [4]. Prior work [4] suggests that reinforcing HS to neutralize the effect of its shedding from the GCX will maintain Cx43 expression and open gap junctional communication. This study determined HS regeneration efficacy by measuring EC-to-EC spread of gap junction-permeable Lucifer Yellow dye as an indicator of the activity level of Cx-containing gap junctions.

Shedding of GCX and its component, HS, as well as alterations in gap junctional communication, have been connected to the onset and the progression of many vascular diseases, including atherosclerosis [39-43].

### Materials and Methods

General Methods. The general design of this investigation is summarized in Table 1.

TABLE 1
Summary of Experimental Design

| | | GLYCOCALYX CONDITIONS | | | | | |
|---|---|---|---|---|---|---|---|
| | | | | Regenerated | | | |
| | | | | | | | HS/S1P |
| | | | | Self-Recovery | HS treated | S1P treated | treated |
| | | Control | Degraded | (24 hrs after | (16 hrs | (16 hrs | (16 hrs after |
| | | Untreated | Hep III | HepIII) | after HepIII) | after HepIII) | HepIII) |
| CULTURE | DMEM + 1% P/S | x | x | x | x | x | x |
| CONDITIONS | 10% FBS | x | x | x | x | x | x |
| PRE-TREATMENT | DMEM + 1% P/S | x | x | x | x | x | x |
| (2 hours) | 1% BSA | x | x | x | x | x | x |
| | 2.5 × 10⁻⁶ Hep III | | x | x | x | x | x |
| POST-TREATMENT | DMEM + 1% P/S | x | x | x | x | x | x |
| | 1% BSA | x | x | x | x | x | x |
| | 59 mg/ml HS | | | | x | | x |
| | 10 μM S1P | | | | | x | x |
| ASSAYS OF EC | HS Expression | x | x | x | x | x | x |
| FUNCTION | Cx43 Expression at EC Borders | x | x | x | x | x | x |
| | Gap Junction Communication | x | x | x | x | x | x |
| | (Dye Transfer) | | | | | | |

Abbreviations: Hep III is heparinase III; HS is heparan sulfate; S1P is sphingosine 1-phosphate; DMEM Dulbecco's Modified Eagle Medium; P/S is penicillin/streptomycin; FBS is fetal bovine serum; BSA is bovine serum albumin; IU is international units; Cx43 is connexin isoforrn 43.
https://doi.org/10.1371/journal.pone.0186116.t001

Cell Culture. Rat fat pad ECs (RFPEC) [44] at passages 20 to 39 were offspring of cells isolated from rat epididymal fat pad [44] and provided by Dr. Mia Thi of Albert Einstein College of Medicine (Bronx, N.Y.). They were used because they exhibit abundant glycocalyx compared to other cell types and also respond to shear stress like other endothelial cells [4, 45]. The RFPECs are immortalized, making it possible to use late passages. RFPEC were seeded on 12-14-mm diameter and 0.13-0.17-mm thick glass coverslips at a seeding density of 15,000-20,000 cells/cm². Cells were cultured in Dulbecco's Modified Eagle Medium (DMEM, Invitrogen, USA) with 1% penicillin-streptomycin (PS) and 10% fetal bovine serum (FBS, Gibco Life Technologies). Cells were maintained in humidity at 37° C. and 5% CO₂. RFPECs reached full confluence in 3 days.

Cultured RFPECs produce thick and robust GCX in this time period. At 3 days post-seeding the RFPECs' culture medium was supplemented with 1% BSA in place of FBS to stabilize the GCX during preservation and immunolabeling (described below). For enzyme treatment experiments, 25 micro-international units per milliliter (μIU/ml) of heparinase III (Hep III; IBEX, Canada) were added to BSA-containing culture media for 2 hours to degrade HS from the GCX [46]. HepIII was washed out, and the wash-out was followed by a 16-hour incubation of RFPECs in regular BSA-containing media. In self-recovery experiments, RFPEC GCX was left to recover for 24 hours after enzyme degradation. The time frame was chosen in accordance with the 20-hour time period that is required for HS restoration to occur on the surface of ECs [47]. For HS recovery experiments, exogenous HS and/or S1P were applied for 16 hours to accommodate the communication test as an assessment of functional glycocalyx recovery. It has been previously shown that, upon external stimulation, induced communication (transfer of gap junction-permeable Lucifer yellow dye) between neighboring endothelial cells is time-dependent, exhibiting a substantial increase in communication in 16 hours compared to a low level of communication in times limited to 5 hours [6]. After HS was degraded with HepIII, it was replaced by a 16-hour feeding of RFPECs with 59 μg/ml exogenous porcine mucosal HS (Celsus, Cincinnati, Ohio), based on published serum concentration of HS [43]. In sphingosine-1-phosphate experiments, culture media containing enzyme was substituted for media containing either 10 μM of S1P (Sigma-Aldrich) or a combination of 59 μg/ml of HS and 10 μM of S1P for 16 hours after enzyme degradation.

Below is described how GCX structure, connexin expression, and gap junctional coupling were characterized in RFPEC with intact GCX, HS degraded GCX, or different modes of GCX repair.

Scanning electron microscopy. RFPECs, still adherent on glass, were fixed for 1 hour in a mixture of 2% paraformaldehyde, 2% glutaraldehyde, and 0.1 μM cacodylate buffer, with or without 0.15% ruthenium red, at a pH of 7.4. After fixation, RFPECs were washed three times at 5- to 10-minute intervals, prior to being incubated for 1 hour in 0.15 μM cacodylate buffer containing 1% osmium tetroxide. Following another cacodylate buffer wash cycle, RFPECs were dehydrated using a graded series of ethanol washes at concentrations of ethanol that included 30%, 50%, 70% and 85% ethanol, and three washes at 100% ethanol, each for 5 minutes. The dehydration series was followed by critical point drying from CO₂. The RFPECs on glass were then attached to sample mounts using double-sided carbon adhesive and coated with 5-nm platinum using a Cressington 208 HR sputter coater. Imaging was performed using a Hitachi S-4800 scanning electron microscope at low accelerating voltage of 3 kV and a magnification of 3000×.

Immunostaining. RFPECs have high affinity to most antibodies that are available for performing immunocytochemistry [46]. For heparan sulfate (HS), RFPEC monolayers were treated with 2% paraformaldehyde/1% glutaraldehyde in phosphate buffered saline (PBS) for fixation, treated with 2% goat serum in PBS to block non-specific ligands, and stained for three nights at 4° C. with a 1:100 10E4-epitope HS antibody (Amsbio).

As specified by the manufacturer, the HS antibody reacts with many types of heparan sulfate. The reactivity of the HS antibody is abolished after treatment with Hep III, indicating antibody specificity [48, 49]. For secondary detection of HS, Alexa Flour 488 conjugated goat anti-mouse IgG/IgM (H+L; Life Technologies) secondary antibody was used. RFPECs to be processed for connexin 43 (Cx43) were fixed in 4% paraformaldehyde, permeabilized in 0.2% Triton X-100 (Fisher), and blocked in 5% goat serum combined with 0.2% Triton X-100. Connexin 43 staining was performed overnight at 4° C. with 1:100 mouse monoclonal Cx43 antibody (Millipore). The manufacturer indicates that anti-Cx43 corresponds to amino acids 131-142 of human Cx43 and is homologous with rat Cx43 (Millipore). Secondary Cx43 detection was done with Alexa Flour 488 conjugated goat anti-mouse IgG. Control RFPEC samples in which anti-HS or anti-Cx antibodies were omitted prior to the application of secondary antibodies did not exhibit immunofluorescence. These controls confirmed that the staining observed in the RFPECs is not an artefact.

Confocal immunofluorescence microscopy imaging and analysis. Alexa Fluor 488 fluorescent RFPECs were mounted with Vectashield containing DAPI (Vector Labs) and imaged with a Zeiss LSM 700 laser scanning confocal microscope. HS and Cx43 were imaged at 63× magnification (oil objective) and 40× magnification (oil objective), respectively. Lasers with excitation wavelengths of 490 nm (for HS or Cx43) and 350 nm (for DAPI) were used to obtain XY-plane slices. Laser gain and transmission were kept below fluorophore saturation levels. Confocal slice intervals were 0.2 μm for HS and 0.7 μm for Cx43. Slices were Z-projected using NIH ImageJ software.

For further HS analysis, from the en face view of the 490-nm channel Z-projection, ImageJ measured and divided the area of fluorescence by the area of the total field of view to obtain a percent value. This percentage represented the amount of RFPECs that was covered by GCX. Cross-sectional images were qualitatively assessed to ensure HS presence on the RFPEC surface and not inside the cells.

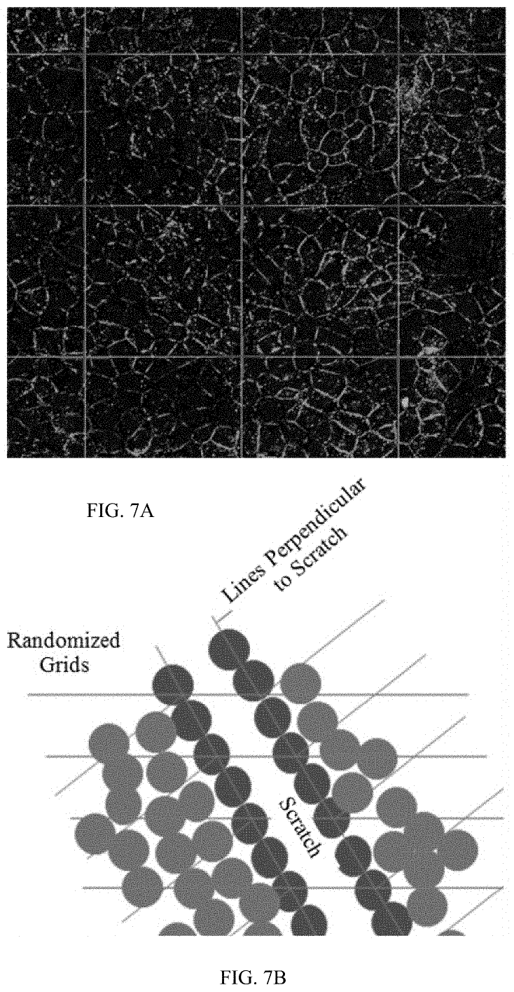

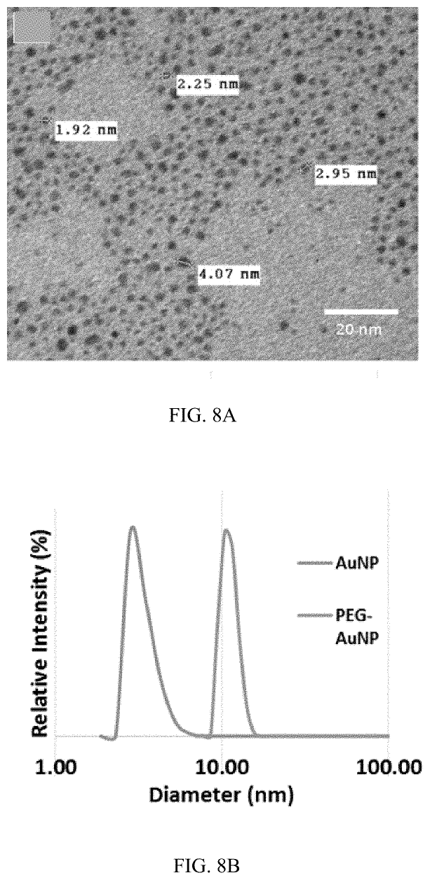

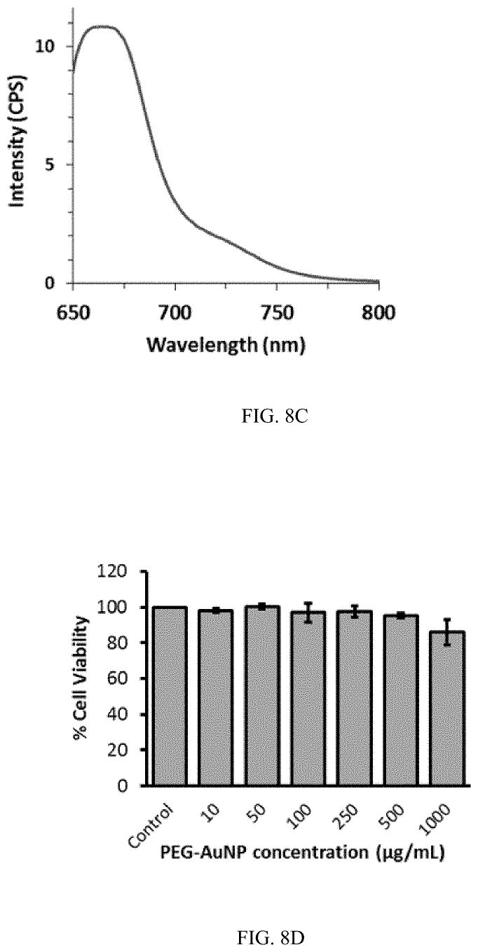

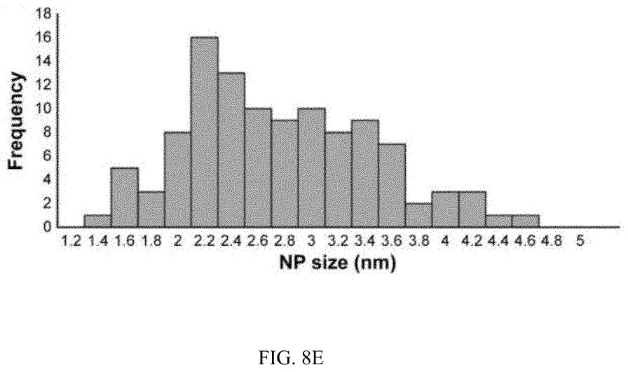

To complete the analysis of Cx43, images of multiple fields were collected to form tiles capturing a 10,0000 μm² field of view. From the en face view of the 490-nm channel Z-projection, ImageJ randomly selected nine cells (FIG. 7A). The ImageJ freehand tool was used to outline the border of each of the nine cells, and to measure each cell perimeter. The length of the perimeter portion that showed Cx43 fluorescence was also measured. The Cx43 length was divided by the perimeter length to determine the percentage of Cx43 distribution along the perimeter of the cell.

Dye transfer assay, fluorescence microscopy and image analysis. Gap junction functionality was assessed by loading Lucifer Yellow in RFPECs, using a scratch technique [50], and observing the extent of Lucifer Yellow transfer from loaded cells to neighboring cells (FIG. 7B). Lucifer Yellow has a molecular weight of 457.3 and can only enter cells via broken membranes or via gap junctions. The scratch loading technique involved pre-incubation of RFPECs (for 1 minute) with calcium- and magnesium-free Hanks' Balanced Salt Solution (HBSS; Life Technology) containing 5 mg/ml Lucifer Yellow dye (Life Technology). A 5-μm diameter tip scribe (Ted Pella, Inc, USA) was then used to carefully create a straight scratch in the RFPEC monolayer and to allow the dye to enter the scratched cells. After the dye was loaded, it spread via open gap junctions to adjacent intact cells, with the extent of Lucifer Yellow spread reaching a plateau by 10 minutes after loading. At that point, excess dye was washed out and RFPECs were imaged at 10× magnification (dry objective) using a Zeiss Observer Z1 fluorescence microscope. From recorded images of dye transfer after scratch loading, ImageJ randomly selected dye spread locations that would be quantified along the scratch. At those random locations, lines perpendicular to the scratch axis were drawn, and the intact fluorescent cells (scratched cells excluded) were counted. The resultant values were taken to represent the extent of Lucifer Yellow spread across the cell monolayer.

Statistics. Data were obtained from 3-5 separate experiments. Per condition, duplicate samples were examined. For HS samples, 5 data points were obtained; for Cx43 samples, 9 data points were obtained; and for Lucifer Yellow samples, 20 data points were obtained. Data was reduced to mean±SEM. GraphPad Prism software was used to analyze the data via one-way ANOVA and Bartlett's statistical correction test, which is sensitive to deviation from normality. Differences in means were statistically significant at p<0.05. Statistical significance of differences in means is specified in the Drawings and the Brief Description of the Drawings.

### Results

HS expression in intact GCX, enzyme degraded GCX, and repaired GCX conditions. Membrane-attached extracellular matrix of RFPECs cultured at passages 20 to 39 on 0.13-0.17-mm thick and 12-14-mm diameter round glass was examined. Expression of the GCX and its HS component on the surface of these RFPECs was confirmed using scanning electron microscopy [51] and immunofluorescence confocal microscopy [28, 46].

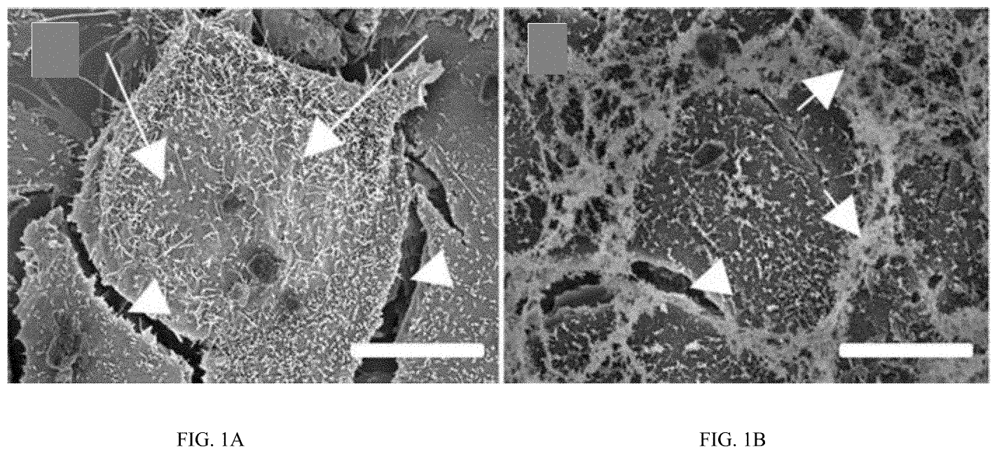

At baseline conditions (untreated control), where RFPECs were fully confluent and had not yet undergone any treatment, scanning electron micrographs distinctly showed long, thin, extracellular microvilli structures extending from the surface of the RFPEC plasma membrane (arrows shown in FIG. 1A). Ruthenium red staining revealed a large portion of other extracellular structures consisting of GAGs, localized on the apical surface as well as at the junction between cells (arrow shown in FIG. 1B). The GCX collapses during the dehydration process that is required for scanning electron microscopy, complicating quantification of GCX structure [28]. Scanning electron microscopy experiments revealed a number of intercellular gaps (arrowheads shown in FIGS. 1A and 1B), which are hypothesized to be due to dehydration and which reinforced that this approach would be useful for GCX localization and structure but not for precise quantification. A correlative microscopy approach that required less detrimental cell preparation, such as immunofluorescence confocal microscopy, would be required [28].

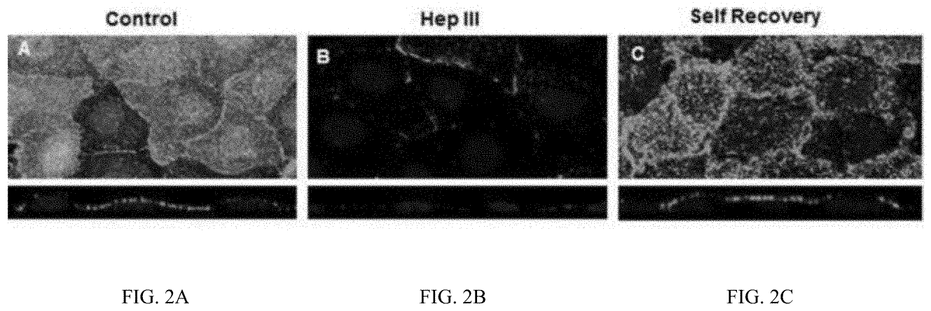

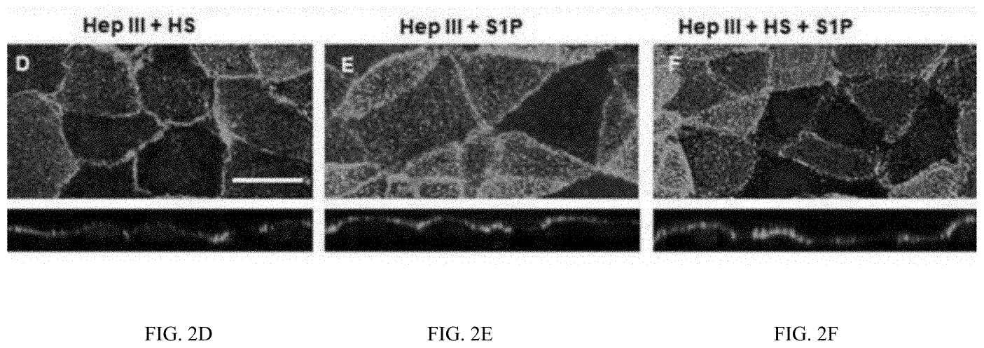

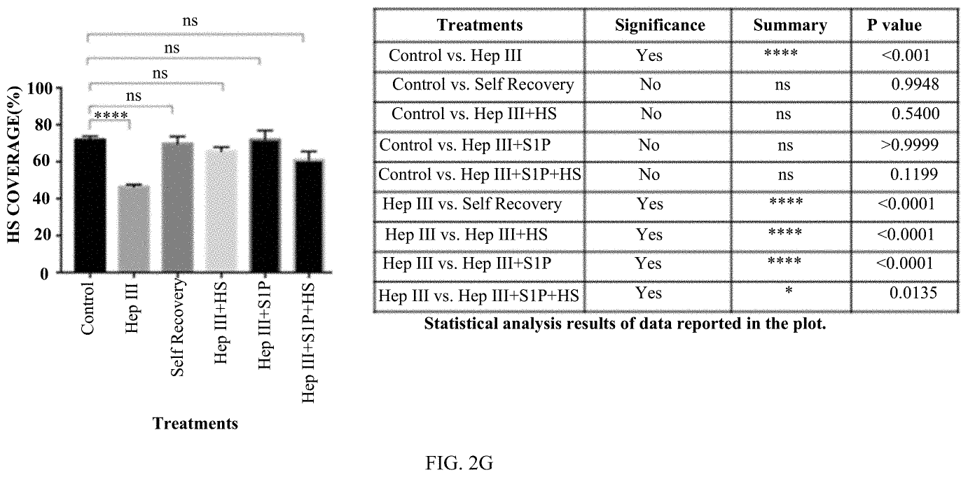

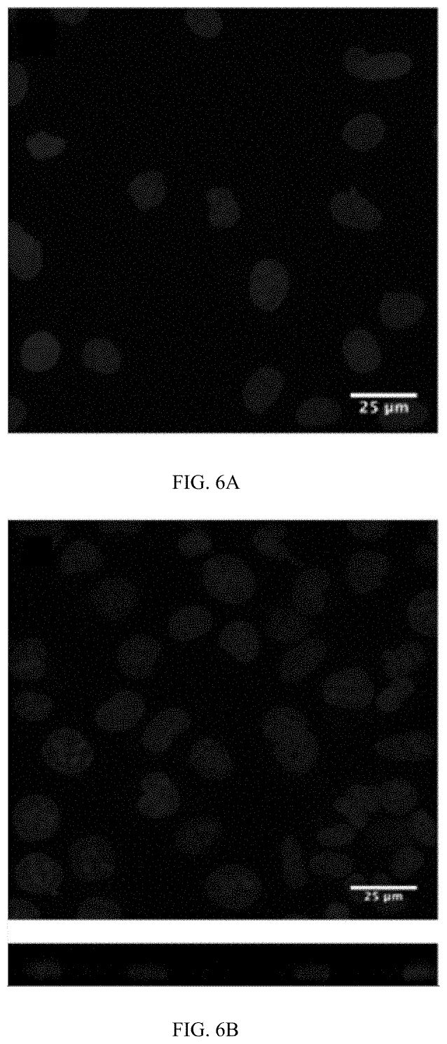

Confocal micrographs illustrated that one of the GAGs on the apical EC surface included HS, as expected (FIGS. 2A-G). For baseline conditions in which RFPEC GCX was left intact and untreated, 71.85±1.90% of cell surfaces were covered with HS, as indicated in immunofluorescence labelling (FIGS. 2A and G). Control RFPEC samples in which HS antibody was omitted prior to the application of secondary antibodies did not exhibit immunofluorescence (FIG. 6A). These controls confirmed that the staining observed in the RFPECs is not artefactual. The effect of enzymatic removal of HS from RFPEC GCX using Hep III is shown in FIG. 2B and quantified in FIG. 2G. RFPECs treated with a 25 IU/ml concentration of Hep III for 2 hours, and probed at 16 hours post-enzyme treatment, showed HS coverage of 46.4±1.20%, which is statistically significantly less than in untreated control samples (FIG. 2G). As a positive control, after enzyme degradation the RFPECs were allowed to recover for 24 hours and self-regenerate their HS coverage. The 24-hour time period for self-regeneration was selected because it is established that HS restoration on the surface of ECs requires 20 hours in static conditions [47]. This cellular self-regeneration resulted in restoration of HS coverage to 69.79±3.92% (FIGS. 2C and G), which was statistically similar to baseline conditions and statistically greater than enzyme treatment conditions (FIGS. 2C and G). It was found that restoration of HS by artificial regeneration could be achieved in a shorter time period (16 hours) than what was required for restoration of HS by cellular self-regeneration. For artificial regeneration of HS, to counteract the effect of Hep III, immediately after Hep III treatment, RFPECs were exposed to 59 μg/ml of exogenous HS during the 16-hour period following enzyme treatment. This caused an increase in the coverage of HS to 65.58±2.26% (FIGS. 2D and G). The added HS was also found to be non-toxic for RFPECs (data not shown) and showed up on the cell surface and was not internalized by the cells (FIG. 2D, orthogonal view). Treatment for 16 hours with 10 μM of S1P to neutralize Hep III yielded HS coverage of 71.97±5.02% (FIGS. 2E and G). Delivering 10 μM S1P in combination with 59 μg/ml exogenous HS for 16 hours resulted in HS coverage of 60.97±4.59%. Statistical analysis confirmed that artificially recovered HS, via the addition of exogenous HS and/or S1P, was significantly more than HS in enzyme-treated conditions and similar to HS in baseline conditions (FIGS. 2F and G).

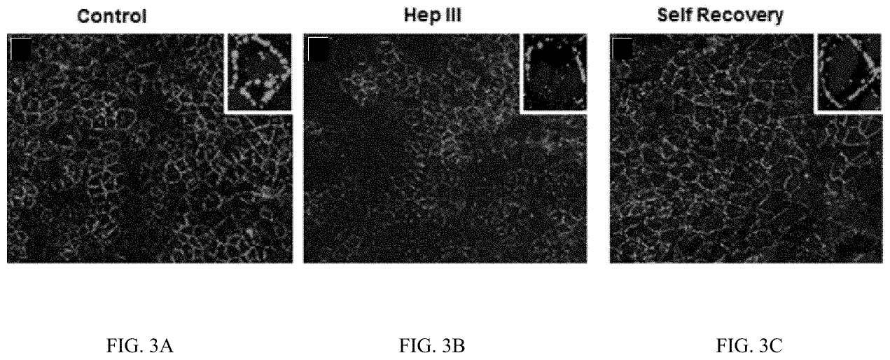

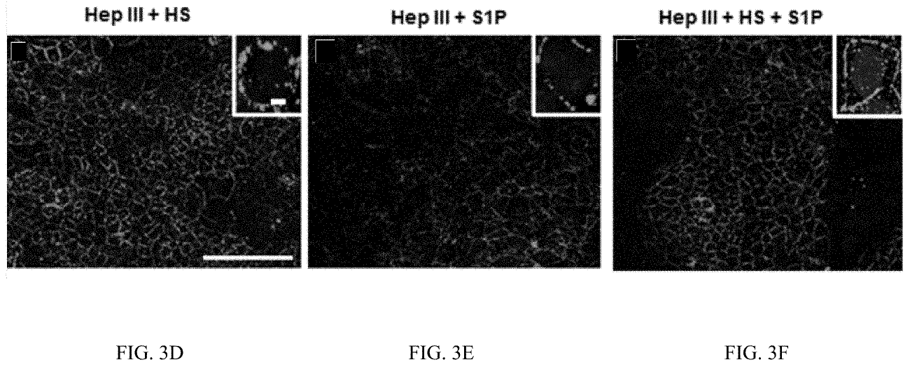

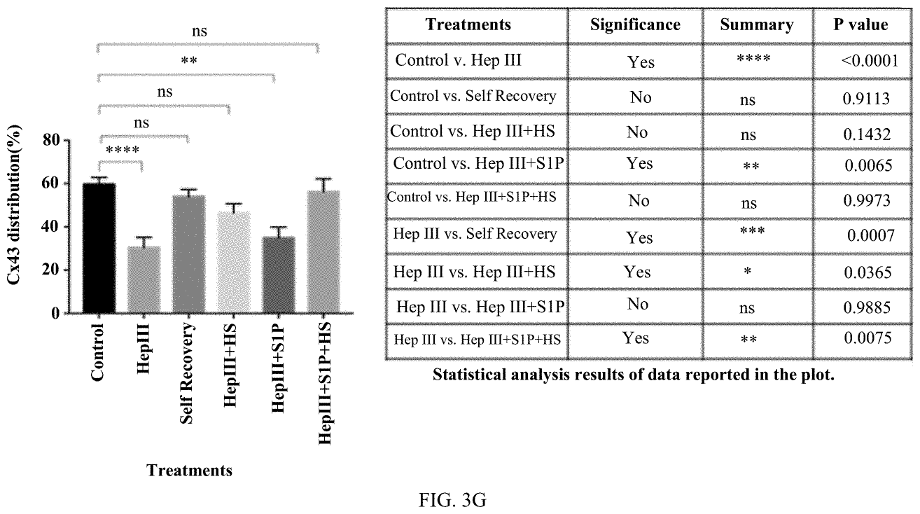

Cx43 in intact GCX, enzyme degraded GCX, and repaired GCX conditions. To study the effect of GCX HS conditions on Cx-containing gap junctions, the distribution of Cx43 at cell borders in samples of EC with intact GCX HS, degraded GCX HS, and GCX HS recovered by various strategies was analyzed. Confocal microscopy studies confirmed that Cx43 is abundantly expressed by RFPECs, and localized primarily at the cell borders where functional gap junctional communication takes place. The initial Cx43 distribution in untreated control RFPEC samples was 59.61±3.20% along the perimeter of the cells (FIGS. 3A and G). Specificity of Cx43 immunolabeling was confirmed by omission of Cx43 antibody and incubation of control RFPEC samples with secondary antibody only, which did not result in any detectable immunofluorescence (FIG. 6A). Enzymatic removal of HS by using 25 IU/ml of Hep III resulted in a statistically significant decrease (p<0.05) in the percentage of Cx43 distribution, to 30.40±4.65% (FIGS. 3B and G), which is approximately 30% less than in untreated control samples (FIGS. 3A and G). In RFPECs that were allowed to self-regenerate their HS coverage after enzymatic degradation, Cx43 appeared along 53.89±3.44% of cell borders, which was statistically equivalent to control conditions and statistically greater than enzyme treatment conditions (FIGS. 3C and G). Addition of 59 μg/ml of HS to counteract enzymatic degradation resulted in Cx43 increase to 46.45±4.21%, representing a statistically significant recovery compared to enzyme-treated samples and statistical similarity to control conditions (FIGS. 3D and G). Samples with S1P treatment after enzyme degradation presented a Cx43 distribution of 34.81±5.16%, which, surprisingly, was not statistically different than enzyme-treated samples and statistically less than baseline levels at control conditions (FIGS. 3E and G). Addition of both HS and S1P resulted in an increase in Cx43 distribution to 56.21±6.32%, a statistically significant recovery from enzyme treatment conditions and statistically close to baseline levels (FIGS. 3F and G). This HS recovery due to combined HS and S1P can be attributed primarily to exogenous HS, since S1P alone could not induce HS recovery.

Gap junction communication is lost with GCX degradation and recovery depends on the mode of GCX repair. Having confirmed Cx expression levels per GCX condition, the extent of functional communication via Cx-expressing gap junctions was measured. The opening and closing of the gap junctions was assessed by end-point quantification of the extent of movement of gap junction-permeable, scratch-loaded Lucifer Yellow dye between neighboring cells, in monolayers of RFPECs with intact GCX and in comparison to monolayers of RFPECs with degraded GCX or after different modes of GCX recovery. In cultured RFPEC monolayers, dye spread (intercellular communication) from the scratch to relatively few neighboring cells was taken to suggest weak communication. Spreading of dye among a relatively large number of cells indicated stronger communication. Dye spread was never observed to extend throughout the entire cell monolayer despite the fact that most RFPECs were expressing Cx43.

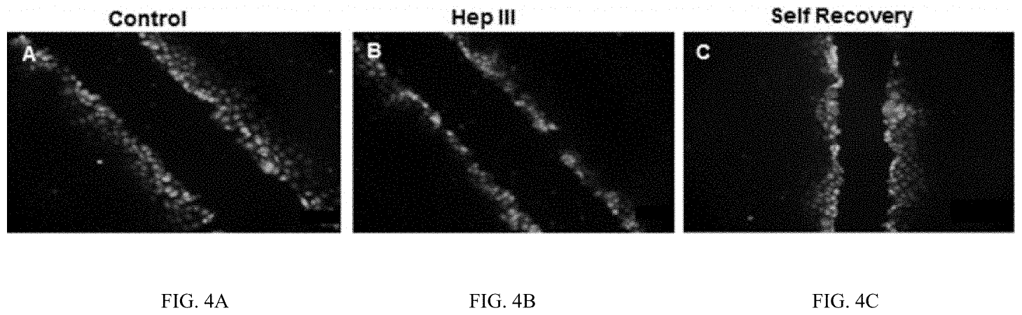

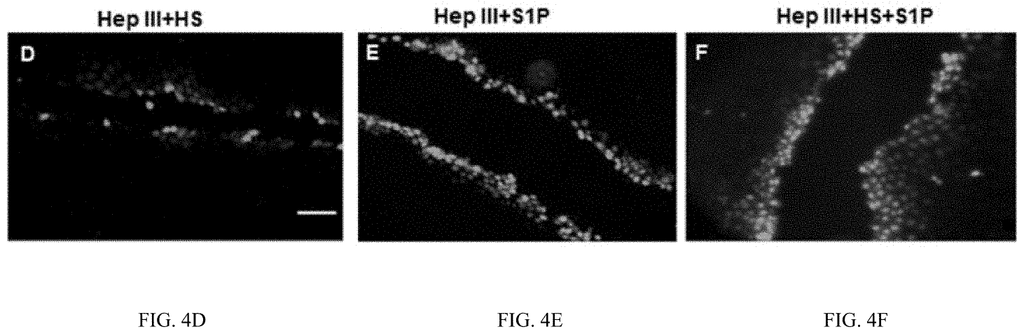

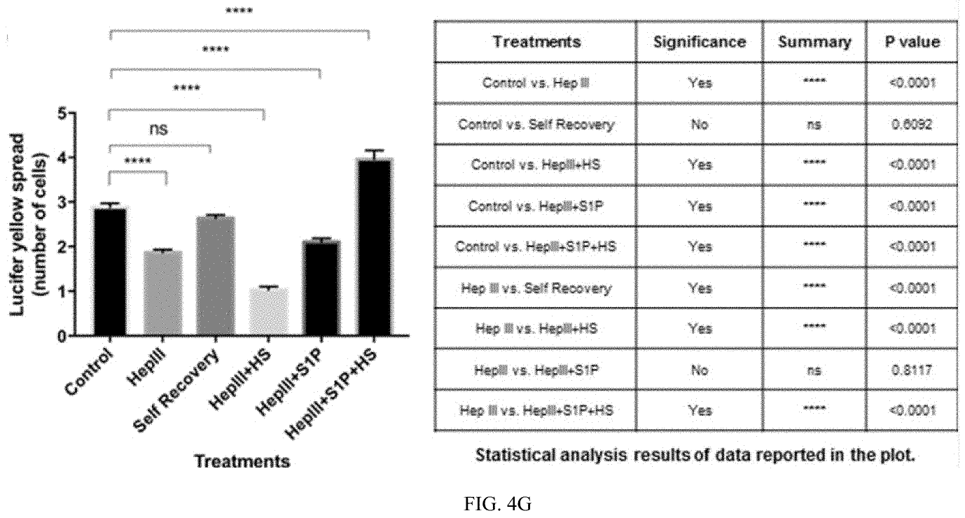

As indicated in FIGS. 4A and G, for every cell along the scratch, uploaded Lucifer Yellow dye transferred to 2.88±0.09 neighboring RFPEC in untreated control conditions. Following Hep III removal of HS from RFPEC, a statistically significant decrease in Lucifer Yellow spread was observed, with 1.87±0.06 cells receiving the dye from an adjacent scratch-loaded cell (FIGS. 4B and G). Self-regeneration of HS after enzyme degradation yielded Lucifer Yellow spread of 2.64±0.07 cells, which was statistically similar to control conditions and statistically significant recovery from enzyme conditions, as expected (FIGS. 4C and G). In RFPECs that had been exposed to HS degradation enzyme, artificial replacement of HS with the exogenous GAG did not yield any recovery of Lucifer Yellow dye-coupling. Surprisingly, under conditions of HS recovery with exogenous HS, scratch-loaded dye was only transferred to 1.03±0.07 cells (FIGS. 4D and G). This was statistically less than dye transfer in both baseline and Hep III conditions (FIGS. 4D and G). Artificial recovery with only S1P added after enzyme degradation showed a Lucifer Yellow dye spread of 2.06±0.08 cells (FIGS. 4E and G), statistically similar to the dye transfer level under Hep III treatment conditions. Pleasingly, after the treatment of RFPEC with combined HS and S1P, 3.96±0.23 cells received Lucifer Yellow dye. This cell number is statistically significantly high when compared to cell number in both untreated control and Hep III-treated conditions (FIGS. 4F and G). This communication data, taken together with the Cx43 data, demonstrates that treatment of ECs with exogenous HS combined with S1P is a good artificial HS regeneration approach for simultaneous recovery of both Cx43 protein and gap junction function.

### Discussion

The study was commenced by creating cell culture models of degraded and regenerated GCX, using RFPECs as an experimental cell culture system because of the inherent RFPEC expression of abundant GCX that contains substantial HS (FIGS. 1A-2G). A model of degraded GCX was created via Hep III-induced HS degradation (FIGS. 2B and G), an approach that is consistent with previous work [40, 46, 47]. This enzymatic degradation model mimics diseased conditions for which release of GAGs from the endothelium surface and into the bloodstream have been reported [43, 52, 53]. The GCX regeneration model used herein, on the other hand, is a new contribution to the GCX research field. It is the first demonstration that endothelial GCX can be considerably restored in vitro by addition of exogenous HS (FIG. 2D). The cell surface GCX capacity to recognize and bind to extracellular GAGs was exploited. The concentration of supplemental HS, 59 μg/ml, was chosen to align with values for the amount of HS found in the arterial blood of patients suffering from global ischemia reported by Rehm et al. [43]. It was anticipated that RFPECs may not incorporate the exogenous HS in a functional manner. An alternative GCX regeneration strategy was to use S1P to both regenerate and stabilize the GCX [25, 26] after enzymatic degradation and regeneration. It was encouraging to find that feeding RFPECs with the prescribed concentration of exogenous HS alone, S1P alone, or HS together with S1P led to renewed HS expression in the RFPEC surface GCX, at a status that matched control conditions. Although using the rat ECs was ideal because of the abundant GCX that could be easily manipulated, this study may be limited by the possibility that the results could be different if other cells were used. For example, using ECs from different vascular beds with potentially different GCX expression patterns could affect the outcomes of this study. Also, primary cell cultures could give different results. Reports of the patterns of HS expression in bovine- and human-derived primary ECs, for example, differ from the RFPEC HS pattern, and are regulated by external mechanical stimulation [54]. With this being said, RFPEC are ideal, because they enable translation of this research to preclinical in vivo studies in the future. Rat animal models have commonly been used for preclinical translational research to test the ability of new drug treatments to mitigate pro-atherosclerotic factors and attenuate atherogenesis in vivo [55]. The RFPEC cultures used in this study are meant to be a species match for the rat animal pre-clinical trials that will be conducted in the future.

GCX importance in key physiological events is well-documented in numerous reports on studies of how characteristic cell functions are gained or lost as a consequence of GCX subcomponent degradation [46, 56, 57]. To date, Thi et al. have been the only group to study GCX regulation of intercellular gap junctional communication [4]. They removed the principal GCX component, HS, and demonstrated gap junction protein desensitization to externally applied flow stimuli that would normally displace the protein. In this study, by successfully degrading and subsequently regenerating HS, a tool was created with which to substantially enhance the body of knowledge about GCX role in inter-endothelial gap junction functionality. Similar to previous findings [4], enzymatic degradation of HS reduced Cx43 at the RFPEC perimeter, where cell-to-cell communication activity takes place, by about 50% compared to baseline conditions. This study is the first to observe significant Cx43 recovery at cell borders, to approximately 80% of baseline conditions, as a result of simple replacement of HS to counteract enzymatic HS degradation. Combinatorial treatment of RFPECs with HS and S1P resulted in further enhanced Cx43 recovery at cell borders, to approximately 95% of baseline conditions. These results emphasize the importance of the GCX as a regulator of cell membrane expression of gap junction protein. The expectation is that similar findings can be obtained for active cell communication, which directly depends on Cx expression and mediates other key EC functions that are prominent in vascular health and disease [32, 58].

In contrast to the Cx43 results, changes in active communication (assessed by Lucifer Yellow dye transfer) did not parallel the experimentally induced changes in HS and the GCX. As was expected, removal of HS from RFPEC reduced the spread of dye from scratch-loaded to neighboring cells by approximately 50%, compared to untreated RFPECs. This reduction was attributed to HS-induced decrease in membrane Cx43 and an associated decline in the number of open gap junction channels. This result further confirmed the fundamental relationship between the GCX and gap junction function. However, with HS regeneration via exogenous HS, gap junction communication remained closed. It is unclear why exogenous HS restoration did not induce gap junction channels to re-open along with renewal of Cx43 expression at cell borders. Without wishing to be bound by any particular theory, it is plausible that GCX restoration by exogenous HS does not translate to full repair of the plasma membrane, which is critical for gap junction proteins to be expressed at the membrane [59] at a level that is sufficient for gap junction formation, because the experimentally restored GCX has a configuration of low stability that exerts reduced tension [60] on the trans-membrane core proteins and, consequently, reduced tension on the cytoskeleton. Downstream, it is possible this low tension may destabilize the cytoskeleton link with the intercellular junction complex. This weak link could result in improper Cx43 alignment and keep connexins from combining to form connexons, block connexon docking to form gap junctions, or prevent gap junction gates from opening. This hypothesis is supported by the results of preliminary studies that actin disruption by cytochalasin D bars cell-to-cell movement of Lucifer Yellow dye and other ions and molecules (data not shown).

The bioactive agent S1P has been reported to preserve GCX [25, 29, 61], and was recently shown to induce recovery of the HS GCX component in the absence of HS at baseline conditions [29]. The detailed mechanism underlying S1P's role in GCX preservation and growth is still under investigation. In addition, S1P has been shown to enhance the strength of the intercellular junction complex in a study that showed S1P causes redistribution of ZO-1 to lamellipodia and cell-to-cell appositions [62], and in another study that demonstrated that S1P enhances the role of Cx in vasculoprotection [63]. Through these reported mechanisms, and possibly others, S1P maintains vascular functions such as regulation of transendothelial permeability [25, 62]. Surprisingly, the use of S1P to regenerate degraded HS did not improve the expression of Cx43 or the level of active communication. Instead, S1P turned out to be an important co-factor for exogenous HS. Regeneration of the GCX via the combination of S1P with exogenous HS resulted in both increased Cx43 at cell borders and greater movement of Lucifer Yellow dye across cells, indicating a recovery of structure and function of both GCX and gap junctions. In fact, HS/S1P-treatment over-recovered gap junctional dye spread, achieving a 1.4-fold increase compared to pre-degradation baseline conditions. Arguably, this gap junction hyperactivity (above baseline level) may signify some form of abnormality [64] or pathology. However, in a recent report by Jiang et al., it was demonstrated that high levels of Cx43-mediated gap junctional communication attenuate the degree of malignancy in multiple cancer cell lines [65]. Considering that cancer and vascular diseases share common pathology progression pathways [66], the results of the study conducted by Jiang et al. can be extrapolated to suggest that over-recovery of interendothelial gap junction-mediated dye spread is not necessarily pathological, and may actually be physiologically beneficial.

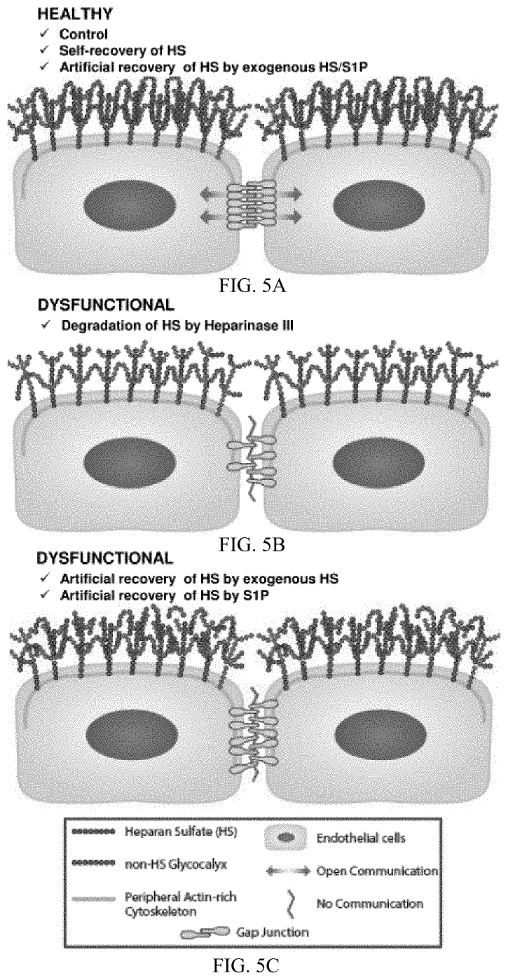

The findings of this work add fundamental knowledge to this understudied area and are encouraging for future therapeutics. As depicted in FIGS. 5A-C, the novel and compelling findings include: (i) removal of GCX-associated HS not only has the ability to alter organization of gap junction proteins, namely Cx43, but it also shuts down gap junction channel activity, and (ii) GCX repair by treating cells with exogenous HS and S1P restores gap junction protein placement, which translates to the reactivation of gap junction channel activity.

This is the first achievement of functional GCX regeneration in ECs in a manner that effectively restores vasculoprotective EC communication within a short time frame. This current finding could translate to therapy for preventing atherosclerosis and motivates future development of new therapies targeted at the GCX to treat vascular disease. In the meantime, in the absence of widely adopted approaches to reinforce the vascular GCX, implementation of the innovative approach described herein in basic cell culture studies, pre-clinical in vivo studies, and, potentially, in clinical settings will be highly significant to advance GCX knowledge and to better address endothelial-dependent disease processes.

### REFERENCES

1. Chiu Jeng-Jiann and Chien Shu, Effects of disturbed flow on vascular endothelium: pathophysiological basis and clinical perspectives. Physiological reviews, 2011. 91(1): p. 327-387. pmid:21248169
2. Ebong Eno E, Lopez-Quintero Sandra V, Rizzo Victor, Spray David C, and Tarbell John M, Shear-induced endothelial NOS activation and remodeling via heparan sulfate, glypican-1, and syndecan-1. Integrative Biology, 2014. 6(3): p. 338-347. pmid:24480876
3. Yao Yu, Rabodzey Aleksandr, and Dewey C Forbes Jr, Glycocalyx modulates the motility and proliferative response of vascular endothelium to fluid shear stress. American journal of physiology. Heart and circulatory physiology, 2007. 293(2): p. H1023-30. pmid: 17468337
4. Thi Mia M, Tarbell John M, Weinbaum Sheldon, and Spray David C, The role of the glycocalyx in reorganization of the actin cytoskeleton under fluid shear stress: a “bumper-car” model. Proceedings of the National Academy of Sciences of the United States of America, 2004. 101(47): p. 16483-16488. pmid:15545600
5. Ebong Eno E and DePaola Natacha, Specificity in the participation of connexin proteins in flow-induced endothelial gap junction communication. Pfliigers Archiv-European Journal of Physiology, 2013. 465(9): p. 1293-1302. pmid:23568367
6. Ebong Eno Essien, Kim Sanghee, and DePaola Natacha, Flow regulates intercellular communication in HAEC by assembling functional Cx40 and Cx37 gap junctional channels. Am J Physiol Heart Circ Physiol, 2006. 290(5): p. H2015-2023. pmid:16361362
7. Fitzgerald M. L., Wang Z., Park P. W., Murphy G., and Bemrnfield M., Shedding of syndecan-1 and -4 ectodomains is regulated by multiple signaling pathways and mediated by a TIMP-3-sensitive metalloproteinase. J Cell Biol, 2000. 148(4): p. 811-24. pmid: 10684261
8. Koo A., Dewey C. F. Jr., and Garcia-Cardena G., Hemodynamic shear stress characteristic of atherosclerosis-resistant regions promotes glycocalyx formation in cultured endothelial cells. Am J Physiol Cell Physiol, 2013. 304(2): p. C137-46. pmid:23114962
9. Paka L., Kako Y., Obunike J. C., and Pillarisetti S., Apolipoprotein E containing high density lipoprotein stimulates endothelial production of heparan sulfate rich in biologically active heparin-like domains. A potential mechanism for the anti-atherogenic actions of vascular apolipoprotein e. J Biol Chem, 1999. 274(8): p. 4816-23. pmid:9988721
10. Vijayagopal P., Figueroa J. E., and Levine E. A., Altered composition and increased endothelial cell proliferative activity of proteoglycans isolated from breast carcinoma. J Surg Oncol, 1998. 68(4): p. 250-4. pmid:9721712
11. Wang Z., Gotte M., Bemrnfield M., and Reizes O., Constitutive and accelerated shedding of murine syndecan-1 is mediated by cleavage of its core protein at a specific juxtamembrane site. Biochemistry, 2005. 44(37): p. 12355-61. pmid:16156648
12. Yanagishita, M and VC Hascall, Cell surface heparan sulfate proteoglycans. 1992.
13. Davies Peter F, Flow-mediated endothelial mechanotransduction. Physiological reviews, 1995. 75(3): p. 519-560. pmid:7624393
14. Cancel Limary M, Ebong Eno E, Solomon Mensah, Hirschberg Carly, and Tarbell John M, Endothelial glycocalyx, apoptosis and inflammation in an atherosclerotic mouse model. Atherosclerosis, 2016. 252: p. 136-146. pmid:27529818
15. Lewis J C, Taylor Richard G, Jones N D, St Clair R W, and Cornhill J F, Endothelial surface characteristics in pigeon coronary artery atherosclerosis. I. Cellular alterations during the initial stages of dietary cholesterol challenge. Laboratory investigation; a journal of technical methods and pathology, 1982. 46(2): p. 123-138. pmid:7062718
16. van den Berg, Bernard M, Jos Spaan A E, Rolf Titia M, and Vink Hans, Atherogenic region and diet diminish glycocalyx dimension and increase intima-to-media ratios at murine carotid artery bifurcation. American Journal of Physiology-Heart and Circulatory Physiology, 2006. 290(2): p. H915-H920. pmid:16155109
17. Tarbell J M and Cancel L M, The glycocalyx and its significance in human medicine. Journal of internal medicine, 2016.
18. Cheng Ming J, Kumar Rajiv, Sridhar Srinivas, Webster Thomas J, and Ebong Eno E, Endothelial glycocalyx conditions influence nanoparticle uptake for passive targeting. International Journal of Nanomedicine, 2016. 11: p. 3305. pmid:27499624
19. Broekhuizen L N, Lemkes B A, Mooij Hans L, Meuwese Marijn C, Verberne Hein, Holleman Frits, Schlingemann Reinier O, Nieuwdorp Max, Erik Stroes S G, and Vink Hans, Effect of sulodexide on endothelial glycocalyx and vascular permeability in patients with type 2 diabetes mellitus. Diabetologia, 2010. 53(12): p. 2646-2655. pmid:20865240
20. Masola V, Zaza G, Onisto M, Lupo A, and Gambaro Giovanni, Glycosaminoglycans, proteoglycans and sulodexide and the endothelium: biological roles and pharmacological effects. Int Angiol, 2014. 33(3): p. 243-54. pmid:24936533
21. Daniels, B. A., Therapeutic Sulfated Polysaccharides, Compositions Thereof, and Methods for Treating Patients. 2014, Google Patents.
22. Harada Naoki and Maeda Masaakira, Chemical structure of antithrombin-active rhamnan sulfate from Monostrom nitidum. Bioscience, biotechnology, and biochemistry, 1998. 62(9): p. 1647-1652. pmid:9805364
23. Maeda Masaakira, Uehara Tutomu, Harada Naoki, Sekiguchi Masayuki, and Hiraoka Atsushi, Heparinoid-active sulphated polysaccharides fromMonostroma nitidum and their distribution in the chlorophyta. Phytochemistry, 1991. 30(11): p. 3611-3614.
24. Illien-Junger Svenja, Grosjean Fabrizio, Laudier Damien M, Vlassara Helen, Striker Gary E, and latridis James C, Combined anti-inflammatory and anti-AGE drug treatments have a protective effect on intervertebral discs in mice with diabetes. PLoS One, 2013. 8(5): p. e64302. pmid:23691192
25. Zhang Lin, Zeng Min, Fan Jie, Tarbell John M., Curry Fitz-Roy E., and Fu Bingmei M., Sphingosine-1-phosphate Maintains Normal Vascular Permeability by Preserving Endothelial Surface Glycocalyx in Intact Microvessels. Microcirculation (New York, N.Y.: 1994), 2016. 23(4): p. 301-310.
26. Abbasi Taimur and Garcia Joe G. N., Sphingolipids in Lung Endothelial Biology and Regulation of Vascular Integrity, in Sphingolipids in Disease, Gulbins E. and Petrache I., Editors. 2013, Springer Vienna: Vienna. p. 201-226.
27. Tarbell J. M. and Ebong E. E., The endothelial glycocalyx: a mechano-sensor and -transducer. Sci Signal, 2008. 1(40): p. pt8. pmid:18840877
28. Ebong E. E., Macaluso F. P., Spray D. C., and Tarbell J. M., Imaging the endothelial glycocalyx in vitro by rapid freezing/freeze substitution transmission electron microscopy. Arterioscler Thromb Vasc Biol, 2011. 31(8): p. 1908-15. pmid:21474821
29. Zeng Y., Liu X. H., Tarbell J., and Fu B., Sphingosine 1-phosphate induced synthesis of glycocalyx on endothelial cells. Exp Cell Res, 2015. 339(1): p. 90-5. pmid:26364737
30. Salmon Andrew H J, Ferguson Joanne K, Burford James L, Gevorgyan Haykanush, Nakano Daisuke, Harper Steven J, Bates David O, and Janos Peti-Peterdi, Loss of the endothelial glycocalyx links albuminuria and vascular dysfunction. Journal of the American Society of Nephrology, 2012. 23(8): p. 1339-1350. pmid:22797190
31. Matsuuchi Linda and Naus Christian C., Gap junction proteins on the move: Connexins, the cytoskeleton and migration. Biochimica et Biophysica Acta (BBA)-Biomembranes, 2013. 1828(1): p. 94-108.
32. Haefliger J. A., Nicod P., and Meda P., Contribution of connexins to the function of the vascular wall. Cardiovasc Res, 2004. 62(2): p. 345-56. pmid:15094354
33. Ampey B. C., Morschauser T. J., Lampe P. D., and Magness R. R., Gap junction regulation of vascular tone: implications of modulatory intercellular communication during gestation. Adv Exp Med Biol, 2014. 814: p. 117-32. pmid:25015806
34. Solan J. L. and Lampe P. D., Connexin43 phosphorylation: structural changes and biological effects. Biochem J, 2009. 419(2): p. 261-72. pmid:19309313
35. Fanning A. S., Jameson B. J., Jesaitis L. A., and Anderson J. M., The tight junction protein ZO-1 establishes a link between the transmembrane protein occludin and the actin cytoskeleton. J Biol Chem, 1998. 273(45): p. 29745-53. pmid:9792688
36. Giepmans B. N. and Moolenaar W. H., The gap junction protein connexin43 interacts with the second PDZ domain of the zona occludens-1 protein. Curr Biol, 1998. 8(16): p. 931-4. pmid:9707407
37. Chen C. H., Mayo J. N., Gourdie R. G., Johnstone S. R., Isakson B. E., and Bearden S. E., The Connexin 43/ZO-1 Complex Regulates Cerebral Endothelial F-actin Architecture and Migration. Am J Physiol Cell Physiol, 2015. 19(00155).
38. Rhett J. M., Jourdan J., and Gourdie R. G., Connexin 43 connexon to gap junction transition is regulated by zonula occludens-1. Mol Biol Cell, 2011. 22(9): p. 1516-28. pmid:21411628
39. Broekhuizen L. N., Mooij H. L., Kastelein J. J., Stroes E. S., Vink H., and Nieuwdorp M., Endothelial glycocalyx as potential diagnostic and therapeutic target in cardiovascular disease. Curr Opin Lipidol, 2009. 20(1): p. 57-62. pmid:19106708
40. Constantinescu A., Spaan J. A., Arkenbout E. K., Vink H., and Vanteeffelen J. W., Degradation of the endothelial glycocalyx is associated with chylomicron leakage in mouse cremaster muscle microcirculation. Thromb Haemost, 2011. 105(5): p. 790-801. pmid:21174004
41. Morel S., Chanson M., Nguyen T. D., Glass A. M., Richani Sarieddine M. Z., Meens M. J., Burnier L., Kwak B. R., and Taffet S. M., Titration of the gap junction protein Connexin43 reduces atherogenesis. Thromb Haemost, 2014. 112(2): p. 390-401. pmid:24828015
42. Nakase T., Ishikawa T., and Miyata H., Protective effects of connexins in atheromatous plaques in patients of carotid artery stenosis. Neuropathology, 2016.
43. Rehm Markus, Bruegger Dirk, Christ Frank, Conzen Peter, Thiel Manfred, Jacob Matthias, Chappell Daniel, Stoeckelhuber Mechthild, Welsch Ulrich, Reichart Bruno, Peter Klaus, and Becker Bernhard F., Shedding of the Endothelial Glycocalyx in Patients Undergoing Major Vascular Surgery With Global and Regional Ischemia. Circulation, 2007. 116(17): p. 1896-1906. pmid:17923576
44. Marcum J A, Rosenberg R D, Heparin Like molecules with anticoagulant activity are synthesized by cultured endothelial cells. 1985: p. 126:365-372.
45. Zeng Y. and Tarbell J. M., The adaptive remodeling of endothelial glycocalyx in response to fluid shear stress. PLoS One, 2014. 9(1): p. e86249. pmid:24465988
46. Zeng Y., Ebong E. E., Fu B. M., and Tarbell J. M., The structural stability of the endothelial glycocalyx after enzymatic removal of glycosaminoglycans. PLoS One, 2012. 7(8): p. e43168. pmid:22905223
47. Giantsos-Adams Kristina M., Koo Andrew Jia-An, Song Sukhyun, Sakai Jiro, Sankaran Jagadish, Shin Jennifer H., Guillermo Garcia-Cardena, and Dewey C. Forbes, Heparan Sulfate Regrowth Profiles Under Laminar Shear Flow Following Enzymatic Degradation. Cellular and Molecular Bioengineering, 2013. 6(2): p. 160-174. pmid:23805169
48. David G., Bai X. M., Van der Schueren B., Cassiman J. J., and Van den Berghe H., Developmental changes in heparan sulfate expression: in situ detection with mAbs. J Cell Biol, 1992. 119(4): p. 961-75. pmid:1385449
49. Bai X. M., Van der Schueren B., Cassiman J. J., Van den Berghe H., and David G., Differential expression of multiple cell-surface heparan sulfate proteoglycans during embryonic tooth development. J Histochem Cytochem, 1994. 42(8): p. 1043-54. pmid:8027524
50. Shu C., Huang W., Zeng Z., He Y., Luo B., Liu H., Li J., and Xu J., Connexin 43 is involved in the sympathetic atrial fibrillation in canine and canine atrial myocytes. Anatol J Cardiol, 2017. 18(1): p. 3-9. pmid:28554986
51. Wu D., Schaffler M. B., Weinbaum S., and Spray D. C., Matrix-dependent adhesion mediates network responses to physiological stimulation of the osteocyte cell process. Proc Natl Acad Sci USA, 2013. 110(29): p. 12096-101. pmid:23818616
52. Mitchell M. J. and King M. R., Physical biology in cancer. 3. The role of cell glycocalyx in vascular transport of circulating tumor cells. Am J Physiol Cell Physiol, 2014. 306(2): p. 16.
53. Broekhuizen Lysette N, Mooij Hans L, Kastelein John J P, Stroes Erik S G, Vink Hans, and Nieuwdorp Max, Endothelial glycocalyx as potential diagnostic and therapeutic target in cardiovascular disease. Current Opinion in Lipidology, 2009. 20(1): p. 57-62. pmid: 19106708
54. Yao Y., Rabodzey A., and Dewey C. F., Glycocalyx modulates the motility and proliferative response of vascular endothelium to fluid shear stress. Am J Physiol Heart Circ Physiol, 2007. 293(2): p. H1023-30. pmid:17468337
55. Cui S., Li W., Lv X., Wang P., Huang G., and Gao Y., Folic acid attenuates homocysteine and enhances antioxidative capacity in atherosclerotic rats. Appl Physiol Nutr Metab, 2017: p. 1-8.
56. Grondahl F., Tveit H., Akslen-Hoel L. K., and Prydz K., Easy HPLC-based separation and quantitation of chondroitin sulphate and hyaluronan disaccharides after chondroitinase ABC treatment. Carbohydr Res, 2011. 346(1): p. 50-7. pmid:21126737
57. Yamagata T., Saito H., Habuchi O., and Suzuki S., Purification and properties of bacterial chondroitinases and chondrosulfatases. J Biol Chem, 1968. 243(7): p. 1523-35. pmid:5647268
58. Joshi Chintamani N and Tulis David A, Connexins and intercellular communication in arterial growth and remodeling. Archives of Physiology, 2015. 2(1): p. 1.
59. Saez J. C., Berthoud V. M., Branes M. C., Martinez A. D., and Beyer E. C., Plasma membrane channels formed by connexins: their regulation and functions. Physiol Rev, 2003. 83(4): p. 1359-400. pmid:14506308
60. Wang N., Naruse K., Stamenovic D., Fredberg J. J., Mijailovich S. M., Tolic-Norrelykke I. M., Polte T., Mannix R., and Ingber D. E., Mechanical behavior in living cells consistent with the tensegrity model. Proc Natl Acad Sci USA, 2001. 98(14): p. 7765-70. pmid: 11438729
61. Zeng Y., Adamson R. H., Curry F. R., and Tarbell J. M., Sphingosine-1-phosphate protects endothelial glycocalyx by inhibiting syndecan-1 shedding. Am J Physiol Heart Circ Physiol, 2014. 306(3): p. H363-72. pmid:24285115
62. Lee Jen-Fu, Zeng Qun, Ozaki Harunobu, Wang Lichun, Hand Arthur R., Hla Timothy, Wang Eugenia, and Lee Menq-Jer, Dual Roles of Tight Junction-associated Protein, Zonula Occludens-1, in Sphingosine 1-Phosphate-mediated Endothelial Chemotaxis and Barrier Integrity. Journal of Biological Chemistry, 2006. 281(39): p. 29190-29200. pmid: 16891661
63. Veenstra R. D., Sphingosine-1-phosphate signals the way for Cx43-mediated cardioprotection. Cardiovasc Res, 2012. 93(1): p. 8-9. pmid:22049533
64. Du Z. J., Cui G. Q., Zhang J., Liu X. M., Zhang Z. H., Jia Q., Ng J. C., Peng C., Bo C. X., and Shao H., Inhibition of gap junction intercellular communication is involved in silica nanoparticles-induced H9c2 cardiomyocytes apoptosis via the mitochondrial pathway. Int J Nanomedicine, 2017. 12: p. 2179-2188. pmid:28356735
65. Jiang G., Dong S., Yu M., Han X., Zheng C., Zhu X., and Tong X., Influence of gap junction intercellular communication composed of connexin 43 on the antineoplastic effect of adriamycin in breast cancer cells. Oncol Lett, 2017. 13(2): p. 857-866. pmid:28356970
66. Ross J. S., Stagliano N. E., Donovan M. J., Breitbart R. E., and Ginsburg G. S., Atherosclerosis and cancer: common molecular pathways of disease development and progression. Ann N Y Acad Sci, 2001. 947: p. 271-92; discussion 292-3. pmid: 11795276

### Example 2. Endothelial Glycocalyx Conditions Influence Nanoparticular Uptake for Passive Targeting

Cardiovascular diseases are facilitated by endothelial cell (EC) dysfunction and coincide with EC glycocalyx coat shedding. These diseases may be prevented by delivering medications to affected vascular regions using circulating nanoparticle (NP) drug carriers. The objective of the present study was to observe how the delivery of 10-nm polyethylene glycol-coated gold NPs (PEG-AuNP) to ECs is impacted by glycocalyx structure on the EC surface. Rat fat pad endothelial cells were chosen for their robust glycocalyx, verified by fluorescent immunolabeling of adsorbed albumin and integrated heparan sulfate (HS) chains. Confocal fluorescent imaging revealed an approximately 3-μm thick glycocalyx layer, covering 75% of the ECs and containing abundant HS. This healthy glycocalyx hindered the uptake of PEG-AuNP as expected because glycocalyx pores are typically 7-nm wide. Additional glycocalyx models tested included: a collapsed glycocalyx obtained by culturing cells in reduced protein media, a degraded glycocalyx obtained by applying heparinase III enzyme to specifically cleave HS, and a recovered glycocalyx obtained by supplementing the media with exogenous HS after enzyme degradation. The collapsed glycocalyx was approximately 2-μm thick with unchanged EC coverage and sustained HS content. The degraded glycocalyx showed similar changes in EC thickness and coverage but its HS thickness was reduced to 0.7-μm and spanned only 10% of the original EC surface. Both dysfunctional models retained six- to seven-fold more PEG-AuNP compared to the healthy glycocalyx. The collapsed glycocalyx permitted NPs to cross the glycocalyx into intracellular spaces, whereas the degraded glycocalyx trapped the PEG-AuNP within the glycocalyx. The repaired glycocalyx model partially restored HS thickness to 1.2 μm and 44% coverage of the ECs, but it was able to reverse the NP uptake back to baseline levels. In summary, this study showed that the glycocalyx structure is critical for NP uptake by ECs and may serve as a passive pathway for delivering NPs to dysfunctional ECs.

### Introduction

The endothelium lines the blood vessel interior and regulates blood vessel stiffness for healthy cardiovascular function.¹ It also acts as a barrier between the blood and underlying tissue by filtering molecules and cells that seek to cross between the two compartments.¹ These are just a few of the known roles of the endothelium. Its proper function relies on its glycocalyx coating that consists of a glycosaminoglycan and proteoglycan mesh layer. This porous structure greatly restricts the passage to plasma proteins, such as albumin of size 7 nm, as well as smaller solutes and water molecules.^{2,3} The glycocalyx glycosaminoglycans, the most common of which is heparan sulfate (HS),⁴⁻⁶ are anchored to endothelial cells (ECs) via transmembrane proteoglycans.⁷ Sialic acid is another glycosaminoglycan of the glycocalyx that is integral in cellular-molecular interactions, while hyaluronic acid and its receptors, such as CD44, are well-characterized and important in cellular function and communication.⁷ The glycosaminoglycans also interact with plasma proteins and other blood-borne molecules, producing variable glycocalyx thicknesses on the outer EC surface.^{2,8} Additionally, the glycocalyx mediates EC signaling and remodeling to provide upstream control of the aforementioned vascular stiffness regulation and filtration roles through its attachment to the EC membrane. As a result, the ECs and the glycocalyx cooperate to exhibit protective and regulatory functions in vascular health.^{1,9-12} Vascular disease progression, such as atherosclerotic vessel hardening or lipid- and macrophage-filled plaque formation, is facilitated by EC dysfunction¹³ and coincides with shedding of the EC glycocalyx coat.¹⁴⁻¹⁷ Furthermore, a compromised glycocalyx has been shown to increase the permeability of nanoscale particles in other cell types such as erythrocytes¹⁸ and cancer tumor cells.¹⁹ Therefore, a potential avenue for treating vascular diseases is to target ECs and their glycocalyx, rather than simply utilizing the systemic and medicinal treatments that are common today.²⁰

EC-specific drug targeting can be enabled with the use of nanoparticle (NP)-based medicine, which has recently been a major focus for researchers.^{21,22} Studies have shown that nanosized particles can be fine-tuned to deliver drugs and control a multitude of scenarios, including extending circulation time in the blood stream and targeting specific organs or sites of disease.^{22,23} Lengthening NP residence times in the blood stream, through delaying clearance by the immune system or filtration by the kidneys, is achieved by covering the NPs with neutrally charged polymer coatings.²³⁻²⁷ While local injection of NP-based materials is available for joint and wound therapy,^{25,28,29} there are several common scenarios that require delivery to difficult-to-reach or unknown locations, such as tumors and metastatic disease. These situations often involve systemic administration through either oral ingestion or, relevant to the present study, venous injection.³⁰ Such approaches require that the surface of NPs be conjugated to targeting ligands to increase the odds of particle accumulation at the desired location.²⁶ This targeting strategy typically leverages the overexpression of enzymes, decrease in pH,^{26,31,32} leakiness of the tissue, and/or enhanced tissue retention of the nanosized particles.^{23,29} These features can be exploited to activate the circulating NP drug carriers at the target organ or disease site.^{33,34} The presence and integrity of the glycocalyx coating of the EC play a critical role in the cellular uptake of circulating NPs. Unfortunately, the glycocalyx is generally not properly modeled in the development of NP-based therapeutic approaches and can lead to incorrect uptake and permeability measurements in vitro.^{18,19,28} Additionally, ECs are not the only cell type to express glycocalyx—most other mammalian cells as well as circulating cancer cells also express unique glycocalyx.^{18,19,28,33,35} Therefore, elucidating the role the glycocalyx plays in the cellular uptake of NPs by ECs will benefit the field of nanomedicine and drug delivery for cardiovascular and other diseases.

This study investigated whether EC exclusion and uptake of polymer-coated NPs are determined by the state of the glycocalyx. The glycocalyx thickness and the extent to which it covers monolayers of EC were measured by fluorescent immunolabeling of glycocalyx components, adsorbed bovine serum albumin (BSA) and HS. This was followed by confocal imaging and analysis, where an untreated intact glycocalyx, protein deficiency-induced collapsed glycocalyx, enzymatically-degraded glycocalyx, and repaired glycocalyx were examined. For these glycocalyx configurations, EC uptake of fluorescent polyethelyne glycol (PEG)-ylated gold NPs (PEG-AuNP)³⁶ was assessed by quantifying and mapping the subcellular locations of the fluorescent signals emitted by the NPs. The exclusion of PEG-AuNP by healthy and recovered glycocalyx and an increased uptake under glycocalyx dysfunction confirm that glycocalyx health is a critical factor for designing NP drug delivery therapeutics.

### Materials and Methods

RFPEC Culture. A rat fat pad endothelial cell (RFPEC) line³⁷ was utilized, originally isolated from rat epididymal fat pads by Dr. Mia Thi of Albert Einstein College of Medicine (Bronx, N.Y., USA). RFPECs were selected for their ability to naturally express a robust glycocalyx.³⁸ These ECs were cultured in tissue culture flasks at 37° C. and 5% CO₂ in a humidified environment with Dulbecco's Modified Eagle's Medium (DMEM) supplemented with 10% fetal bovine serum (FBS) and 1% penicillin-streptomycin, all purchased from Thermo Fisher Scientific (Waltham, Mass., USA). For 3-(4,5-dimethylthiazol-2-yl)-5-(3-carboxymethoxyphenyl)-2-(4-sulfophenyl)-2H-tetrazolium (MTS) assays (Sigma-Aldrich Co., St Louis, Mo., USA), RFPECs were seeded at 3.7×10⁴ cells/cm² in tissue culture-treated, 96-well plates (Falcon; Thermo Fisher Scientific) and imaged on a SpectraMax M3 plate reader to show cell viability and metabolic activity. For confocal imaging, RFPECs were seeded onto 12-mm diameter round glass cover slips (Thermo Fisher Scientific) at 1×10⁴ cells/cm² in tissue culture-treated six-well plates (Falcon; Thermo Fisher Scientific). In both cases, the cells were allowed to grow for 48-60 hours to full confluence before starting the experiment.

PEG-AuNP. Gold was chosen to be the core of the NPs for its inert property and its widespread use in other applications, such as imaging under magnetic resonance imaging, surface-enhanced Raman scattering, and radiotherapy.³⁹⁻⁴¹ AuNP functionalized with a polyethylene glycol (PEG) corona were synthesized following the protocols reported by Kumar et al.³⁶ Following the formation of tetrakis-(hydroxymethyl)-phosphonium chloride-coated AuNP (Sigma-Aldrich Co.), three types of functional PEG were added onto its surface: SH-PEG-OCH₃, SH-PEG-COOH, and SH-PEG-NH₂ (Laysan Bio, Arab, Ala., USA) at a ratio of 1:1:2 using gold-thiol chemistry. Addition of the functional PEG was confirmed using Fourier transform infrared spectroscopy (Bruker Vertex-70; Bruker Optik GmbH, Ettlingen, Germany). The variety of functional PEG allows for additional customization in future iterations of the NPs. After dialysis in 12-14 kDa molecular weight cutoff tubes (EMD Millipore, Billerica, Mass., USA) and lyophilization, an Alexa Fluor 647 N-hydroxysuccinimide (NHS) ester (succinimidyl ester) was conjugated onto the NH₂ functional groups on the AuNP to allow for visualization under fluorescence. NP size was determined using a transmission electron microscope (TEM) (JEOL JEM-1000, Tokyo, Japan) at 80 kV and 100,000× magnification and dynamic light scattering (DLS) (Brookhaven 90Plus Particle Size Analyzer; Brookhaven Instruments Corporation, Holtsville, N.Y., USA). PEG-AuNP fluorescence activity was confirmed using a Jobin Yvon Fluoromax 4.

RFPEC glycocalyx treatments. Table 2 summarizes how the RFPECs were cultured to generate various glycocalyx configurations. For control glycocalyx conditions, RFPEC cultures were grown in DMEM with 10% FBS and 1% penicillin-streptomycin. To generate dysfunctional glycocalyx conditions, the regular culture media of other RFPEC cultures was replaced with two variations of the modified media. For the collapsed glycocalyx configuration, the cells were grown in low serum media that consisted of DMEM with 1% penicillin-streptomycin and only 2% FBS.¹ For the degraded glycocalyx configuration, cultures were pretreated with DMEM/10^{%} FBS/1% penicillin-streptomycin containing a 2.5×10⁻⁶ IU heparinase III (Hep III) (IBEX Pharmaceutical, Montreal, QC, Canada) enzyme for 2 hours, selectively degrading the HS glycosaminoglycans of the glycocalyx. To simulate a repaired glycocalyx state, the media containing Hep III was replaced with exogenous HS (Celsus Laboratories, Cincinnati, Ohio, USA) dissolved in DMEM/10% FBS/1% penicillin-streptomycin at 59 μg/mL. All RFPEC cultures, including those with untreated, collapsed, degraded, or repaired glycocalyx configurations, were exposed to 550 μg/mL of lyophilized and ultraviolet-sterilized PEG-AuNP during the 16-hour treatment time. Afterward, the free-floating particles were gently washed out before fixation to ensure that only the AuNP entrapped within the glycocalyx would be imaged. The red fluorescence detected from conjugated Alexa Fluor 647 was attributed to AuNP within the RFPEC monolayers (details are given in the “Confocal imaging and analysis” section).

TABLE 2
Treatment details and media composition
for glycocalyx configurations.

| | Glycocalyx configurations | | | |
|---|---|---|---|---|
| | | | | Repair |
| | Control | Collapse | Degraded | (Hep III + |
| | (untreated) | (low serum) | (Hep III) | HS) |
| Culture | | | | |
| DMEM + 1% P/S | X | X | X | X |
| 10% FBS | X | X | X | X |
| Pretreatment | | | | |
| DMEM + 1% P/S | X | X | X | X |
| 10% FBS | X | | X | X |
| 2% FBS | | X | | |
| 2.5 × 10⁻⁶ IU Hep III | | | X | X |
| Treatment | | | | |
| DMEM + 1% P/S | X | X | X | X |
| 10% FBS | X | | X | X |
| 2% FBS | | X | | |
| 59 μg/mL HS | | | | X |
| 550 μg/mL | X | X | X | X |
| PEG-AuNP | | | | |

Note:
The X is to indicate the component on the left hand column is present within each of the configuration media.
Abbreviations: HEP III, heparinase III; HS, heparan sulfate: DMEM, Dubecco's Modified Eagle's Medium; P/S, penicillin/streptomycin; FBS, fetal bovine serum; PEG-AuNP, PEGylated gold NPs.

Immunostaining. To visualize the glycocalyx, BSA from the media or the HS glycosaminoglycan was fluorescently tagged through immunostaining. RFPECs were first rinsed in phosphate-buffered saline (PBS) (Corning Incorporated, Corning, N.Y., USA) containing Ca²⁺ and Mg²⁺ adjusted to a pH of 7.2. RFPEC slides were then fixed with 2% paraformaldehyde/0.1% gluteraldehyde (Electron Microscopy Sciences, Hatfield, Pa., USA) in PBS for 30 minutes. PBS was used to thoroughly wash the fixatives before the slides were blocked with 2% goat serum (Sigma-Aldrich Co.) diluted in PBS for 30 minutes. Then, 0.02 μg/μL of the primary antibody in PBS containing 2% goat serum was introduced and left in a humidity-controlled chamber at 4° C. overnight for BSA staining, or for three nights for HS staining. The primary BSA stain utilized a rabbit IgG anti-albumin (bovine serum), obtained from Thermo Fisher Scientific, and the primary HS stain used a 10E4 epitope HS mouse monoclonal IgM antibody from Amsbio (Abingdon, UK). Following incubation with primary antibody, RFPECs were washed again in PBS and then incubated in 0.002 μg/L of secondary antibody in PBS containing 2% goat serum for 30 minutes at room temperature and in the dark. Alexa Fluor 488 goat anti-rabbit IgG and Alexa Fluor 488 goat anti-mouse IgG, IgM fractions (Thermo Fisher Scientific) were used for the secondary detection of BSA and HS stains, respectively. After incubation with secondary antibody, the RFPECs were washed in PBS and then mounted onto glass microscope slides (Thermo Fisher Scientific) using VECTASHIELD mounting medium (Vector Laboratories, Burlingame, Calif., USA). The mounting medium contained 4′,6-diamidino-2-phenylindole (DAPI), a fluorescent stain that binds to adenine and thymine regions of DNA to mark the cell nuclei. Generic nail polish was used to seal the slides before imaging.

Confocal imaging and analysis. A Zeiss LSM 700 confocal microscope (Carl Zeiss Meditec AG, Jena, Germany) using a 63× oil immersion objective with excitation wavelengths of 350, 490, and 650 nm were used to obtain XY-plane slices of the nuclei of RFPECs, fluorescent glycocalyx (BSA and HS), and PEG-AuNP within RFPECs, respectively. Slices were captured at intervals of 0.2 μm and assembled into Z-projections using NIH ImageJ software (ImageJ version 1.49k; National Institute of Health, USA). Glycocalyx coverage of RFPEC layers was quantified using ImageJ to analyze the en face view of the Z-projections of green channel (490 nm). The fluorescence threshold was adjusted to eliminate the background noise and then converted to black and white such that the green signal became white and the background signal was reduced to black. ImageJ measured the percentage of white areas compared to the total field of view in the micrograph, which was then equated to RFPEC glycocalyx coverage. Glycocalyx thickness was also measured with the help of ImageJ based on cross sections (orthogonal views) of the Z-projections of green channel. Three evenly distributed locations on the surface of each RFPEC were randomly selected by ImageJ. Lines perpendicular to the RFPEC surface were drawn at each point from the base of the glycocalyx to the top of the glycocalyx, as indicated by the green fluorescence. The length of the lines was measured and averaged to determine glycocalyx thickness.

It is important to note that BSA immunofluorescence was taken as an indicator of the structure of the whole glycocalyx, as albumin from the FBS in culture media is adsorbed throughout the glycocalyx. Therefore, it is reasonable to assume that BSA thickness and coverage measurements are equal to the overall glycocalyx thickness and coverage. On the other hand, HS immunofluorescence revealed thickness and coverage for the HS component only within the glycocalyx mesh, allowing for a more close examination during HS degradation and recovery. Red fluorescence, as detected by ImageJ, was attributed to clusters of the PEG-AuNP trapped within the glycocalyx.³⁶ To quantify the amount of PEG-AuNP present in each sample, Z-stacks were projected en face and the number of red pixels was measured in ImageJ using the measure area tool. Additionally, orthogonal views were used to map the distributions of regions where the PEG-AuNP localized within the cells by using the plot profile function in ImageJ and correlating the height of red fluorescence in relation to glycocalyx position. DAPI staining of RFPEC nuclei was not quantified and only served to mark the RFPEC.

Statistics. NP uptake values, glycocalyx thickness, and percent coverage measurements are expressed as mean±standard error of the mean. These measurements were taken from five independent experiments. For each condition studied, two samples were studied per experiment. From each sample, a minimum of five confocal Z-stacks were collected. Data set groups were individually compared against the data set of the untreated control group. The difference between the groups was analyzed for statistical significance using an unpaired Student's t-test with an alpha value of 0.05.

### Results and Discussion

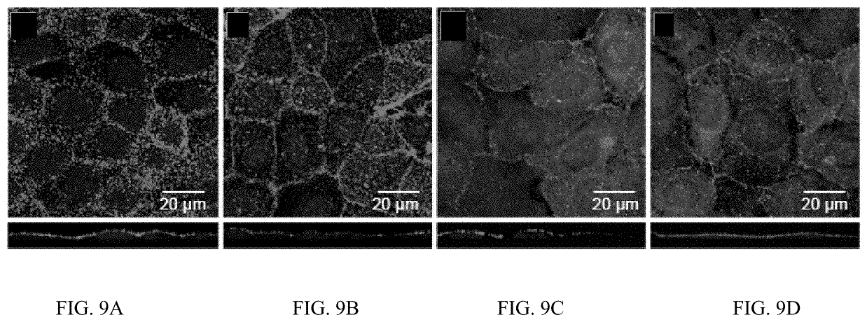

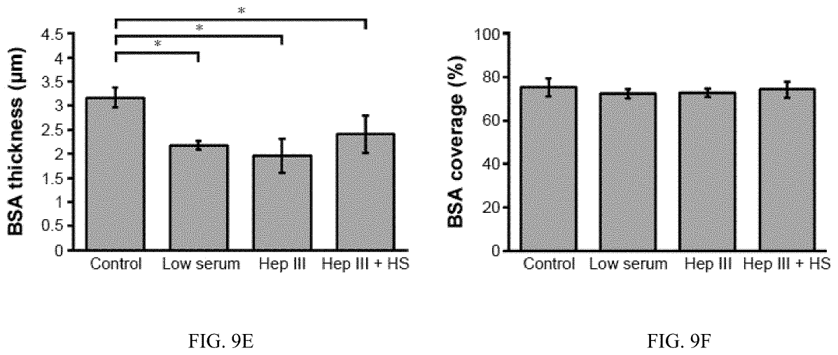

Characterization of fluorescent, PEG-AuNP. Synthesized AuNP were imaged under TEM and DLS, giving a diameter of 2.8±0.8 and 3.0±0.6 nm, respectively (FIGS. 9A, B and E). After PEGylation, the diameter of the particles increased to 10.6±1.2 nm (FIG. 9B). Furthermore, the particles were confirmed to have a fluorescence emission peak at approximately 667 nm (FIG. 9C) and insignificant toxicity to RFPECs up to 1 mg/mL (FIG. 9D). From zeta potential calculations, particle charge was found to be −14 mV (data not shown). The size of the PEG-AuNP is ideal for observing NP-EC interactions because of the 7-nm porosity of the glycocalyx¹, as well as the negatively charged glycocalyx surface. A healthy glycocalyx should prevent these particles from becoming entrapped, but once the glycocalyx is compromised and the barrier function is impaired, the PEG-AuNP will be able to pass through and taken up into the cells. The Alexa Fluor 647 probes were imaged alongside the healthy, collapsed, degraded, and regenerated glycocalyx samples to observe total AuNP uptake as well as the distribution within the endothelial monolayer.

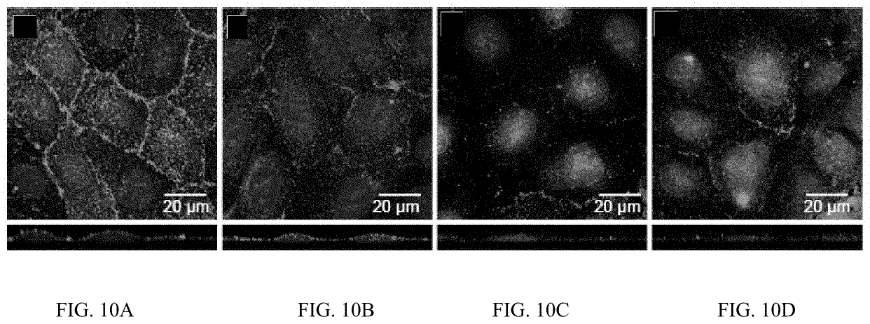

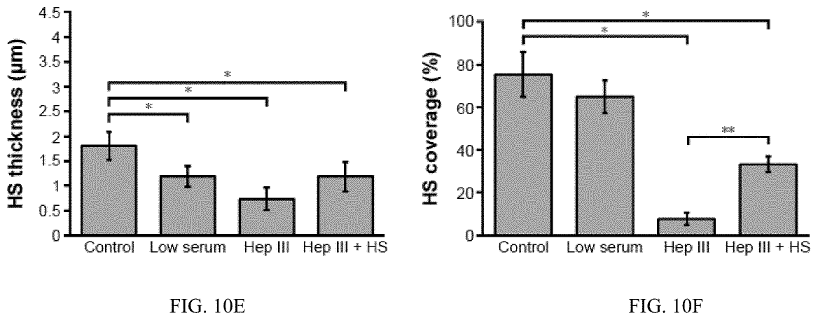

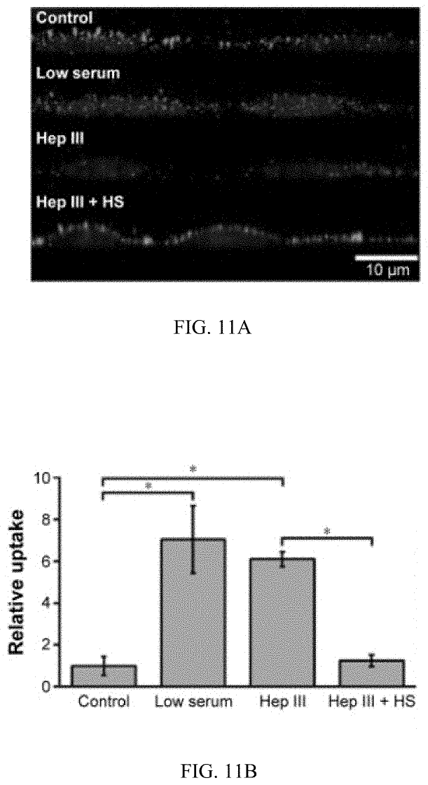

Healthy glyocalyx excludes PEG-AuNP from ECs. ECs with an intact and healthy glycocalyx were modeled by RFPECs and their unmanipulated glycocalyx. As reported by others,^{37,42} RFPECs grown in static culture express a robust glycocalyx, unlike other ECs whose glycocalyx development is hindered in static conditions. The dimensions of healthy RFPEC glycocalyx were obtained by measuring BSA coverage and thickness on the EC surface. As previously mentioned, the albumin from the culture media is adsorbed throughout the expanse of the glycocalyx, which gives it its full thickness.⁴³ From confocal measurements of BSA immunofluorescence, the total glycocalyx in healthy conditions was found to have a thickness of 3.2±0.2 μm, and cover 75.2%+4.0% (FIGS. 9A-F) of the EC layer surface. Upon immunostaining and measurement of the HS subcomponent of the glycocalyx, it was found to be 1.8±0.2 μm thick, and to cover 75.2%+10.6% of the RFPEC surface (FIGS. 10A-F). As the most abundant glycosaminoglycan, HS appears to be integrated within the glycocalyx mesh close to the cell surface, as seen by the adjacent nuclei from the DAPI stain within the cross sections (FIGS. 10A-F). Previous studies suggest that the outer region of the glycocalyx consists largely of hyaluronan and sialic acid, which are two other important components of the glycocalyx.^{10,18,34,44} These glycocalyx elements were not explored in the present study, although their roles and the roles of other glycosaminoglycans in EC permeability is a question of great interest. The dimensions of total glycocalyx (BSA) and the HS subcomponent measured were within the range of previously reported values for rodent cell cultures and animal models using the same fixation and microscopy approach.^{37,38,45} Therefore, these results provide strong indication that the untreated RFPEC glycocalyx is healthy; owing to its substantial thickness and continuity, the RFPEC glycocalyx mesh is expected to serve as a robust, molecular sieve as seen in the body.

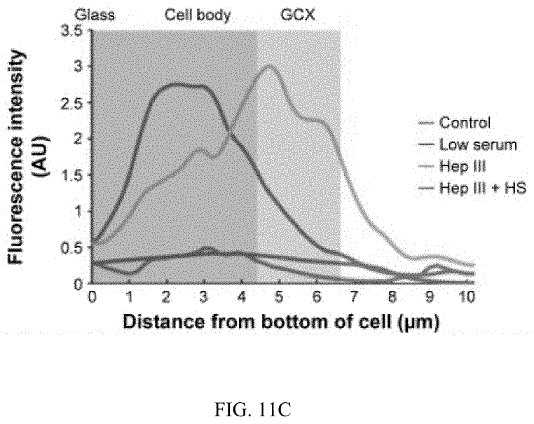

To confirm the barrier function of a healthy RFPEC glycocalyx, the extent of uptake of 10-nm diameter PEG-AuNP³⁶ was measured by the red fluorescence emitted by the conjugated Alexa Fluor 647. It was found to be strikingly low with two or three clusters of PEG-AuNP per cross section (FIG. 11A), covering 1% of the image (assessed by counting pixels) and presenting a normalized value of about 1 in FIG. 11B. The appearance of red particles in clusters can be attributed to the particles' tendency to aggregate after entrapment. The low uptake of these NP clusters demonstrates that, in healthy conditions, the glycocalyx does not allow many 10 nm NPs to penetrate into the endothelium. This NP exclusion is likely due to the 7-nm pore size of the glycocalyx. In addition, the NPs are negatively charged (−14 mV), which causes the particles to be repelled by the negatively charged glycocalyx. Identification of the red fluorescence location within the monolayer showed an even dispersion, indicating no specific localization within the cells (FIG. 11C) for the small number of particles present.

Glycocalyx dysfunctions lead to distinct PEG-AuNP uptake. Next, the glycocalyx dysfunction treatments were applied to the EC to observe whether NP permeability was influenced by changes in the glycocalyx. The first model of dysfunctional glycocalyx was in the collapsed form, generated by culturing RFPEC in a serum-deficient setting containing 2% FBS rather than the normal 10%. For a collapsed glycocalyx, due to low serum treatment, total glycocalyx (BSA) thickness decreased by 31% from 3.2±0.2 to 2.2±0.1 μm (FIGS. 9B and E). On the other hand, the coverage of the whole glycocalyx was not significantly affected at 72.3%+2.0% compared to the control (75%) (FIG. 10F). Serum-starved, collapsed glycocalyx measurements for HS were comparable to those of the whole glycocalyx where HS thickness decreased by 34% to 1.19±0.2 μm (FIGS. 10B and E), but its coverage of EC was not affected significantly at 64.8%+7.6% (FIG. 10F).

The second dysfunctional model of degraded glycocalyx was achieved through exposure of RFPEC to heparinase III for selective degradation of the HS component. Heparinase III treatment resulted in a whole glycocalyx (BSA) thickness of 1.96±0.3 μm (FIGS. 9C and E), a 38% decrease from the control measurement of 3.2±0.2 m. Heparinase III slightly disrupted the continuity of the whole glycocalyx, resulting in the coverage of 72.7%+1.9% (FIGS. 9C and E) of the EC layer, a decrease of 3.4%. The persisting structural stability of the whole glycocalyx, even after the enzymatic removal of individual glycosaminoglycans has been previously reported.³⁸ However, the results from the current heparinase III experiment produced a decrease in total glycocalyx thickness, but statistically negligible change in the continuity of the total glycocalyx. The discrepancy between previous and presently reported measurements may have been due to the treatment time scale. The RFPECs in this study were exposed to 2 hours of enzyme treatment followed by another 16 hours of incubation with NPs to assess the uptake. Previously, RFPECs were exposed to an enzyme for a short 2-hour period before immediately preserving the cells and immunostaining.³⁸ The present approach yielded a significantly thinner matrix while preserving the continuity of the total glycocalyx matrix. Focusing on the major glycocalyx subcomponent, HS, its thickness was statistically significantly decreased by 59% from 1.8±0.2 to 0.74±0.2 μm (FIGS. 10C and E). This was expected due to the specific action of heparinase III on HS, and resulted in a substantial 89.8% decrease in HS coverage from 75.2%+10.6% to 7.7%+2.9% (FIGS. 10C and F).

The thinning of the glycocalyx through dysfunction may result in an increased permeability of the endothelial glycocalyx. Wiesinger et al. reported an association between sickness and thinning of the glycocalyx, both in vivo (in septic mice) and in vitro (in human) ECs exposed to sepsis-associated mediators.⁴⁶ Further, Yang et al. reported correlation between the thickness of cancer cell glycocalyx and the uptake of gold nanocapsules, suggesting that a thinner glycocalyx allows more uptake.¹⁹ This suggests that the models utilized in this study, which have significantly reduced glycocalyx thickness, should lead to an impaired barrier function and a greater particle uptake. For both collapsed and degraded glycocalyx configurations, statistically significantly higher uptake of Alexa Fluor 647-conjugated PEG-AuNP was observed as expected. Compared to the control RFPEC monolayers with intact glycocalyx, RFPECs with collapsed glycocalyx took up 7.0±1.6-fold more NPs, whereas RFPECs with degraded glycocalyx took up 6.1±0.4-fold more, as indicated by the fluorescence intensity quantification (FIGS. 11A and B). Additionally, the two dysfunctional glycocalyx layers exhibited different AuNP distribution within the ECs: collapse due to the lack of serum which led to more AuNP being internalized within the cell body, while enzyme degradation showed AuNP primarily localizing within the glycocalyx (FIG. 11C). Consistent results over five experiments suggested that the NP internalization that was observed is global and that the subcellular localization of the AuNP is statistically relevant. While others have reported a similar increase in nanoscale particle permeability for other cell types and conditions,^{18,34,47,48} this is the first time the experiment has been conducted with nonactively targeted AuNP in RFPECs with the intracellular localization identified. Additionally, previously published studies did not attempt to repair the glycocalyx, but only compared the healthy glycocalyx to the degraded glycocalyx.^{18,23,34} Hence, exogenous HS was added back into the culture after HS degradation, in the hopes of observing a change in PEG-AuNP uptake from the degraded model.

HS replacement restores glycocalyx blockage of PEG-AuNP entry in the EC. Recovery of the impaired permeability from the degradation of the HS glycosaminoglycans was attempted by incubating the heparinase III-treated cells in media containing exogenous HS (DMEM with 59 μg/mL HS, 10% FBS, and 1% penicillin-streptomycin) for 16 hours before fixation. The HS supplementation increased the overall glycocalyx thickness from 1.96±0.3 to 2.41±0.3 μm, but it did not affect the overall glycocalyx coverage, which remained at 75.2%+4.0% (FIGS. 9D and E). The HS component thickness was also partially restored, from 0.74±0.23 to 1.21±0.31 μm, but not quite reaching the baseline thickness of 1.81±0.28 μm (FIGS. 10D and E). More importantly, the replacement increased HS coverage to 33.1%+3.7% (FIGS. 10D and F). Compared to the healthy control coverage of 75.2%+10.6% and degraded coverage of 7.7%+2.9%, it is a significant improvement from 10% to 44% of the baseline measurements (FIGS. 10D and F). These results show a partial structural recovery of the glycocalyx layer with respect to HS after incubation of ECs with the exogenous HS-supplemented media.

There are several possible explanations for the observed partial but not complete HS and glycocalyx recovery after adding HS. First, the concentration of HS used in the rat EC culture media was equal to HS concentrations measured in plasma from human clinical trials.⁴⁹ HS found in the human plasma should correlate to the amount of HS shed from the clinical trial participants' blood vessel walls, and may not necessarily correlate to the HS degraded in this study. Additionally, HS concentration is different between species and concentration imbalance of sugars in the media can cause further glycocalyx damage.^{37,50} Quantification of HS produced by RFPECs and the amount shed through heparinase III treatment will improve dosage calculations for future experiments. Second, the exogenous HS was derived from porcine, which may cause a mismatch of physical characteristics with the RFPEC culture. The glycosaminoglycans differ from species to species not only in concentrations but also in patterns of sulfate groups on the sugar chains.^{5,6} The porcine HS may not be fully compatible with the RFPECs, and alludes to the partial recovery,⁵¹ and in future studies the use of rat-derived HS may improve glycocalyx regrowth. Third, there is little control over how the exogenous HS is incorporated into the glycocalyx after addition to the culture. It can bind to proteoglycan sites that are exposed after enzyme cleavage, be entrapped within the glycocalyx mesh in a manner similar to albumin, or be endocytosed to increase the intracellular concentration of HS components rather than the cell surface concentration of HS. In the endogenous HS production pathway, the proteoglycans are synthesized in the ribosome and glycosylated in the Golgi apparatus, which are then exported to the cell membrane via Golgi- and rough endoplasmic reticulum-regulated secretory vesicles.⁵² In future studies, it may be more advantageous to upregulate this endogenous HS production pathway via delivery of plasmids, rather than an exogenous route, to ensure the production of new HS at the cell membrane for a full glycocalyx recovery.

Despite the observed partial HS and glycocalyx recovery after adding exogenous HS, the barrier function was completely recovered with respect to 10-nm NPs. PEG-AuNP uptake studies in the glycocalyx-regenerated EC samples exhibited a staggering decrease in permeability compared to the two dysfunctional glycocalyx models (FIG. 11A). The uptake of the NPs by these cells was measured to be a mere 1.3±0.3-fold greater than the uptake by cells with healthy, intact glycocalyx (FIG. 11B), and the confocal images showed almost no PEG-AuNP entrapment in the monolayer (FIG. 11A). This fold change was significantly low compared to the Hep III-treated samples, demonstrating restored impedance of NP uptake into ECs in the presence of exogenous HS. Similar to the control, the localization plot showed a nonspecific distribution within the RFPEC culture (FIG. 11C). While there are reports of increased NP uptake or interaction for dysfunctional or compromised glycocalyx on various cell types, none have recovered and reexamined the glycocalyx permeability. Although the glycocalyx was not thoroughly regenerated, this recovery of the barrier function points to a new approach in passive NP delivery to ECs within the circulatory system. Design of NP therapeutics such that they take advantage of this permeability changes under dysfunction in conjunction with active targeting can lead to higher targeting efficiency to the areas of interest and bypass the healthy vasculature.

### CONCLUSIONS

Glycocalyx collapse and discontinuity have both been suggested to colocalize with vascular sites that are prone to cardiovascular disease.⁵³ This study focused on taking glycocalyx conditions into consideration for scenarios of targeted, systemic NP-based drug delivery. Glycocalyx structure was modulated by serum deprivation or enzymatic cleavage of HS and subsequently supplementing exogenous HS, and probed using PEG-AuNP transport. The data show that the glycocalyx structure greatly affects the ability of PEG-AuNP to permeate ECs. In a healthy EC culture, the uptake of 10-nm particles was impeded by the endothelial glycocalyx. The dysfunction of the glycocalyx, both collapsed and degraded, led to an increase in NP uptake of over six-fold (FIGS. 11A-C). This indicates a compromised barrier function of the glycocalyx and an additional mechanism for targeting NPs to vascular locations at risk for cardiovascular disease. Furthermore, the recovery of glycocalyx and the associated restoration of the protective function shown by the decrease in PEG-AuNP fluorescence indicate possibilities for nanomedicine-based glycocalyx therapeutics. In addition to the size constraint of the nanocarrier design, regeneration of the glycocalyx can lead to particle rejection once healed, allowing the particles to target other locations downstream. While the AuNPs do not support encapsulation and delivery of glycosaminoglycans, other types of particles can be formulated to specifically repair diseased glycocalyx. For example, physically delivering missing components or upregulating glycocalyx production via gene delivery are potential considerations. Glycocalyx repair would then prevent further particles from attaching and the remaining circulating particles can move downstream to another area of dysfunction—this allows for a better distribution among the various diseased EC areas in vasculature. The PEG-AuNP used also have the potential to be improved by conjugating the functional PEGs with antibodies against endothelial-specific targets (e.g., nonglycocalyx targets, in this case), therapeutics that need to be delivered via the vasculature in an endothelial-specific manner, and imaging probes to enable in situ visualization of NPs. The ability to deliver these NPs to dysfunctional and disease-prone ECs by exploiting glycocalyx behavior will significantly advance cardiovascular and other areas of drug delivery.

### REFERENCES

1. Fels J, Jeggle P, Liashkovich I, Peters W, Oberleithner H. Nanomechanics of vascular endothelium. Cell Tissue Res. 2014; 355(3):727-737.
2. Pries A R, Secomb T W, Gaehtgens P. The endothelial surface layer. Pflugers Arch. 2000; 440(5):653-666.
3. Weinbaum S, Zhang X, Han Y, Vink H, Cowin S C. Mechanotransduction and flow across the endothelial glycocalyx. Proc Natl Acad Sci USA. 2003; 100(13):7988-7995.
4. Gamse G, Fromme H G, Kresse H. Metabolism of sulfated glycosaminoglycans in cultured endothelial cells and smooth muscle cells from bovine aorta. Biochim Biophys Acta.
1978; 544(3):514-528.
5. Ihrcke N S, Wrenshall L E, Lindman B J, Platt J L. Role of heparan sulfate in immune system-blood vessel interactions. Immunol Today. 1993; 14(10):500-505.
6. Oohira A, Wight T N, Bornstein P. Sulfated proteoglycans synthesized by vascular endothelial cells in culture. J Biol Chem. 1983; 258(3):2014-2021.
7. Tarbell J M, Ebong E E. The endothelial glycocalyx: a mechano-sensor and -transducer. Sci Signal. 2008; 1(40):1-8.
8. Adamson R H, Clough G. Plasma proteins modify the endothelial cell glycocalyx of frog mesenteric microvessels. J Physiol. 1992; 445:473-486.
9. Loeven M A, Rops A L, Berden J H, Daha M R, Rabelink T J, van der Vlag J. The role of heparan sulfate as determining pathogenic factor in complement factor H-associated diseases. Mol Immunol. 2015; 63(2):203-208.
10. Reitsma S, Slaaf D W, Vink H, van Zandvoort M, oude Egbrink M G A. The endothelial glycocalyx: composition, functions, and visualization. Pflugers Arch. 2007; 454(3):345-359.
11. Alphonsus C S, Rodseth R N. The endothelial glycocalyx: a review of the vascular barrier. Anaesthesia. 2014; 69(7):777-784.
12. Oberleithner H. Vascular endothelium: a vulnerable transit zone for merciless sodium. Nephrol Dial Transplant. 2014; 29(2):240-246.
13. Tabas I, Garcia-Cardena G, Owens G K. Recent insights into the cellular biology of atherosclerosis. J Cell Biol. 2015; 209(1):13-22.
14. Broekhuizen L N, Mooij H L, Kastelein J J, Stroes E S, Vink H, Nieuwdorp M. Endothelial glycocalyx as potential diagnostic and therapeutic target in cardiovascular disease. Curr Opin Lipidol. 2009; 20(1):57-62.
15. Nieuwdorp M, Meuwese M C, Vink H, Hoekstra J B, Kastelein J J, Stroes E S. The endothelial glycocalyx: a potential barrier between health and vascular disease. Curr Opin Lipidol. 2005; 16(5):507-511.
16. van den Berg B M, Nieuwdorp M, Stroes E S, Vink H. Glycocalyx and endothelial (dys) function: from mice to men. Pharmacol Rep. 2006; 58(Suppl):75-80.
17. Van Teeffelen J W, Brands J, Stroes E S, Vink H. Endothelial glycocalyx: sweet shield of blood vessels. Trends Cardiovasc Med. 2007; 17(3):101-105.
18. Atukorale P U, Yang Y S, Bekdemir A, et al. Influence of the glycocalyx and plasma membrane composition on amphiphilic gold nanoparticle association with erythrocytes. Nanoscale. 2015; 7(26):11420-11432.
19. Yang Y S, Carney R P, Stellacci F, Irvine D J. Enhancing radiotherapy by lipid nanocapsule-mediated delivery of amphiphilic gold nanoparticles to intracellular membranes. ACS Nano. 2014; 8(9):8992-9002.
20. Smith S C Jr, Benjamin E J, Bonow R O, et al. AHA/ACCF Secondary Prevention and Risk Reduction Therapy for Patients with Coronary and other Atherosclerotic Vascular Disease: 2011 update: a guideline from the American Heart Association and American College of Cardiology Foundation. Circulation. 2011; 124(22):2458-2473.
21. Zhang L, Gu F X, Chan J M, Wang A Z, Langer R S, Farokhzad O C. Nanoparticles in medicine: therapeutic applications and developments. Clin Pharmacol Ther. 2008; 83(5):761-769.
22. Kim B H, Hackett M J, Park J, Hyeon T. Synthesis, characterization, and application of ultrasmall nanoparticles. Chem Materials. 2014; 26(1):59-71.
23. Brigger I, Dubernet C, Couvreur P. Nanoparticles in cancer therapy and diagnosis. Adv Drug Deliv Rev. 2002; 54(5):631-651.
24. Adak A K, Li B Y, Lin C C. Advances in multifunctional glycosylated nanomaterials: preparation and applications in glycoscience. Carbohydr Res. 2015; 405:2-12.
25. Garrett I R, Gutierrez G E, Rossini G, et al. Locally delivered lovastatin nanoparticles enhance fracture healing in rats. J Orthop Res. 2007; 25(10): 1351-1357.
26. Li H, Yu S S, Miteva M, et al. Matrix metalloproteinase responsive, proximity-activated polymeric nanoparticles for siRNA delivery. Adv Funct Mater. 2013; 23(24):3040-3052.
27. Torchilin V P. Multifunctional nanocarriers. Adv Drug Deliv Rev. 2012; 64(Supplement): 302-315.
28. Freeman C G, Parish C R, Knox K J, et al. The accumulation of circulating histones on heparan sulphate in the capillary glycocalyx of the lungs. Biomaterials. 2013; 34(22):5670-5676.
29. Lu B, Xiong S-B, Yang H, Yin X-D, Chao R-B. Solid lipid nanoparticles of mitoxantrone for local injection against breast cancer and its lymph node metastases. Eur J Pharm Sci. 2006; 28(1-2):86-95.
30. Chono S, Li S-D, Conwell C C, Huang L. An efficient and low immunostimulatory nanoparticle formulation for systemic siRNA delivery to the tumor. J Control Release.
2008; 131(1):64-69.
31. Oishi M, Hayashi H, Iijima M, Nagasaki Y. Endosomal release and intracellular delivery of anticancer drugs using pH-sensitive PEGylated nanogels. J Mater Chem. 2007; 17(35):3720-3725.
32. Nishimura Y, Takeda K, Ezawa R, Ishii J, Ogino C, Kondo A. A display of pH-sensitive fusogenic GALA peptide facilitates endosomal escape from a bio-nanocapsule via an endocytic uptake pathway. JNanobiotechnol. 2014; 12:11.
33. Paszek M J, DuFort C C, Rossier O, et al. The cancer glycocalyx mechanically primes integrin-mediated growth and survival. Nature. 2014; 511(7509):319-325.
34. Yang C, Liu Y, He Y, et al. The use of HA oligosaccharide-loaded nanoparticles to breach the endogenous hyaluronan glycocalyx for breast cancer therapy. Biomaterials. 2013; 34(28):6829-6838.
35. Lange T, Samatov T R, Tonevitsky A G, Schumacher U. Importance of altered glycoprotein-bound N- and O-glycans for epithelial-to-mesenchymal transition and adhesion of cancer cells. Carbohydr Res. 2014; 389:39-45.
36. Kumar R, Korideck H, Ngwa W, et al. Third generation gold nanoplatform optimized for radiation therapy. Transl Cancer Res. 2013; 2(4):228-239.
37. Ebong E E, Macaluso F P, Spray D C, Tarbell J M. Imaging the endothelial glycocalyx in vitro by rapid freezing/freeze substitution transmission electron microscopy. Arterioscler Thromb Vasc Biol. 2011; 31(8):1908-1915.
38. Zeng Y, Ebong E E, Fu B M, Tarbell J M. The structural stability of the endothelial glycocalyx after enzymatic removal of glycosaminoglycans. PLoS One. 2012; 7(8):e43168.
39. Hainfeld J F, Dilmanian F A, Slatkin D N, Smilowitz H M. Radiotherapy enhancement with gold nanoparticles. J Pharm Pharmacol. 2008; 60(8):977-985.
40. Popovtzer R, Agrawal A, Kotov N A, et al. Targeted gold nanoparticles enable molecular CT imaging of cancer. Nano Lett. 2008; 8(12):4593-4596.
41. Shukla R, Bansal V, Chaudhary M, Basu A, Bhonde R R, Sastry M. Biocompatibility of gold nanoparticles and their endocytotic fate inside the cellular compartment: a microscopic overview. Langmuir. 2005; 21(23):10644-10654.
42. Zeng Y, Tarbell J M. The adaptive remodeling of endothelial glycocalyx in response to fluid shear stress. PLoS One. 2014; 9(1):e86249.
43. Schneeberger E E, Hamelin M. Interaction of serum proteins with lung endothelial glycocalyx: its effect on endothelial permeability. Am J Physiol Heart Circul Physiol. 1984; 247(2):H206-H217.
44. Henry C B, Duling B R. Permeation of the luminal capillary glycocalyx is determined by hyaluronan. Am J Physiol. 1999; 277(2 Pt 2):H508-H514.
45. van den Burg M M, Eizema K, de Graaf-Roelfsema E, et al. Effects of acute exercise and long-term exercise on total Na+, K+-ATPase content and Na+, K+-ATPase isoform expression profile in equine muscle. Am J Vet Res. 2009; 70(7):895-901.
46. Wiesinger A, Peters W, Chappell D, et al. Nanomechanics of the endothelial glycocalyx in experimental sepsis. PLoS One. 2013; 8(11):e80905.
47. Liu J, Agrawal N J, Calderon A, Ayyaswamy P S, Eckmann D M, Radhakrishnan R. Multivalent binding of nanocarrier to endothelial cells under shear flow. Biophys J. 2011; 101(2):319-326.
48. Zhu J, Xue J, Guo Z, Zhang L, Marchant R E. Biomimetic glycoliposomes as nanocarriers for targeting P-selectin on activated platelets. Bioconjug Chem. 2007; 18(5):1366-1369.
49. Rehm M, Bruegger D, Christ F, et al. Shedding of the endothelial glycocalyx in patients undergoing major vascular surgery with global and regional ischemia. Circulation. 2007; 116(17):1896-1906.
50. Piest M, Ankone M, Engbersen J F. Carbohydrate-interactive pDNA and siRNA gene vectors based on boronic acid functionalized poly(amido amine)s. J Control Release. 2013; 169(3):266-275.
51. Ernst S, Langer R, Cooney C L, Sasisekharan R. Enzymatic degradation of glycosaminoglycans. Crit Rev Biochem Mol Biol. 1995; 30(5):387-444.
52. Varki A, Cummings R D, Esko J D. Essentials of Glycobiology. Cold Spring Harbor, N.Y.: Cold Spring Harbor Laboratory Press; 2009.
53. Lewis J C, Taylor R G, Jones N D, St Clair R W, Cornhill J F. Endothelial surface characteristics in pigeon coronary artery atherosclerosis. I. Cellular alterations during the initial stages of dietary cholesterol challenge. Lab Invest. 1982; 46(2): 123-138.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A composition, comprising heparan sulfate, or a pharmaceutically acceptable salt thereof, and sphingosine-1-phosphate, or a pharmaceutically acceptable salt thereof.

2. The composition of claim 1, wherein the heparan sulfate is exogenous heparan sulfate.

3. The composition of claim 2, wherein the heparan sulfate is porcine mucosal heparan sulfate with an average molecular weight of about 15 kDa.

4. A method of regenerating glycocalyx of a cell, comprising contacting the cell with an effective amount of heparan sulfate, or a pharmaceutically acceptable salt thereof, and an effective amount of sphingosine-1-phosphate, or a pharmaceutically acceptable salt thereof.

5-9. (canceled)

10. The method of claim 4, wherein the cell is an endothelial cell.

11. A method of treating a vascular disease in a subject in need thereof, comprising administering to the subject a therapeutically effective amount of heparan sulfate, or a pharmaceutically acceptable salt thereof, and a therapeutically effective amount of sphingosine-1-phosphate, or a pharmaceutically acceptable salt thereof.

12. The method of claim 11, wherein the vascular disease is atherosclerosis, a blood clot, a stroke, peripheral artery disease, an aneurysm, a pulmonary embolism, carotid artery disease, arteriovenous malformation, critical limb ischemia, deep vein thrombosis, chronic venous insufficiency, a varicose vein, coronary artery disease, Raynaud's disease or vasculitis.

13. The method of claim 12, wherein the vascular disease is atherosclerosis.

14. The method of claim 4, wherein the heparan sulfate is exogenous heparan sulfate.

15. The method of claim 14, wherein the heparan sulfate is porcine mucosal heparan sulfate with an average molecular weight of about 15 kDa.

16. An implant, comprising the composition of claim 1.

17. The implant of claim 16, wherein the implant is a stent or catheter.

18. The implant of claim 16, wherein the heparan sulfate, or a pharmaceutically acceptable salt thereof, and the sphingosine-1-phosphate, or a pharmaceutically acceptable salt thereof, are coated on a surface of the implant.

19. The implant of claim 16, wherein the heparan sulfate is porcine mucosal heparan sulfate with an average molecular weight of about 15 kDa.

20. A vascular graft, comprising the composition of claim 1.

21. The vascular graft of claim 20, wherein the heparan sulfate is porcine mucosal heparan sulfate with an average molecular weight of about 15 kDa.

22-24. (canceled)
